(19) United States
(12) Patent Application Publication
(10) Pub. No.: US 2024/0197850 A1
(43) Pub. Date: Jun. 20, 2024

(54) SALMONELLA VACCINE

(71) Applicant: University of Maryland, College Park, College Park, MD (US)

(72) Inventors: Debabrata BISWAS, Rockville, MD (US); Jungsoo JOO, Clarksburg, MD (US); Mengfei PENG, Montgomery Village, MD (US)

(21) Appl. No.: 18/553,686

(22) PCT Filed: Apr. 5, 2022
(86) PCT No.: PCT/US2022/023517

§ 371 Date: Oct. 2, 2023

Related U.S. Application Data
(60) Provisional application No. 63/171,067, filed on Apr. 5, 2021.

(51) Int. Cl. A61K 39/112; A61K 9/00; A61K 39/00; A61P 31/04; C12N 1/20; C12R 1/42

(52) U.S. Cl. CPC A61K 39/0275; A61K 9/0053; A61P 31/04; C12N 1/205; A61K 2039/522; A61K 2039/542; A61K 2039/552; C12R 2001/42

(57) ABSTRACT

Provided are modified bacteria and methods of using the modified bacteria for prophylaxis or treatment of bacterial infections. The modified bacteria contain one or more genomic modifications such that the genomes of the bacteria are altered to encode and produce a holin protein and to encode and produce a lysozyme. The modified bacteria are illustrated using a type of Salmonella enterica (SE) in the form of autolytic SE serovar Typhimurium (S. typhimurium).

### CROSS-REFERENCE TO RELATED APPLICATIONS

This application claims priority to U.S. provisional patent application No. 63/171,067, filed Apr. 5, 2021, the disclosure of which is incorporated herein by reference in its entirety.

### STATEMENT REGARDING FEDERALLY SPONSORED RESEARCH

This invention was made with government support under 20196703029564 awarded by the United States Department of Agriculture (USDA). The government has certain rights in the invention.

### SEQUENCE LISTING

The instant application contains a Sequence Listing which has been submitted electronically in ASCII format and is hereby incorporated by reference in its entirety. Said ASCII file, created on Apr. 5, 2022, is named UMD_Salmonella_Vaccine_ST25.txt, and is 11,819 bytes in size.

### FIELD

The disclosure relates generally to modified bacteria and uses thereof as vaccines against bacterial infections.

### BACKGROUND

Foodborne microbial pathogens induce human enteric diseases, which are recognized as the primary worldwide concern for food safety (Goodwin & Shiptsova, 2002). Salmonella enterica (SE) is a major foodborne pathogen which causes numerous outbreaks, hospitalization, death, and a huge economic burden in developed nations including the United States (Basler, Nguyen, Anderson, Hancock, & Behravesh, 2016). Salmonella infects humans via contaminated foods, specifically chicken meat and eggs. Among the various serovars of SE, Typhimurium and Enteritidis are the top two serovars which are responsible for human infection and colonized in the chicken gut intestine as a gut commensal flora (Ricke, Dunkley, & Durant, 2013). Therefore, control of S. Typhimurium and S. Enteritidis colonization in chicken can reduce the risk of cross-contamination at both pre-harvest and post-harvest levels of the production processes of chicken meat and eggs and is a potential way to reduce foodborne infections with SE.

Currently, attenuated Salmonella vaccine, including AviPro® MEGAN®VAC 1, is available which can slightly reduce the colonization of SE serovars, however, its effectiveness is not satisfactory due to the limited number of antigen-specific antibody developments (Michel, Clermont, Denamur, & Tenaillon, 2010) in in vitro condition. Therefore, a large number of target antigens are utilized in the host immune system. Thus, there is an ongoing and unmet need for improved compositions and methods to protect against SE. The present disclosure is pertinent to this need.

### BRIEF SUMMARY

The present disclosure provides modified bacteria and methods of using the modified bacteria for prophylaxis or treatment of bacterial infections. The modified bacteria contain one or more genomic modifications such that the genomes of the bacteria are altered to encode and produce a holin protein and to encode and produce a lysozyme. The compositions and methods are suitable for use with humans and non-human animals.

In certain examples the modified bacteria are non-pathogenic to the individual to which the modified bacteria are administered. The modified bacteria may be rendered non-pathogenic by modification, or the modified bacteria may be non-pathogenic prior to being modified. In embodiments the modified bacteria are Salmonella. In embodiments, the modified bacteria non-pathogenic Salmonella. In some embodiments, the non-pathogenic bacteria are Salmonella enterica (SE). In some embodiments, the SE are SE intracellular autolytic SE serovar Typhimurium (S. typhimurium).

The holin protein and the lysozyme proteins are not particularly limited and may be encoded by, for example, any phage genome. In certain examples, the holin protein is encoded by gene 13 of Salmonella typhimurium-specific bacteriophage P22, and/or the lysozyme is encoded by gene 19 of the S. typhimurium-specific bacteriophage P22. In one example, the holin protein is encoded by gene 13 of Salmonella typhimurium-specific bacteriophage P22 and the lysozyme is encoded by gene 19 of the S. typhimurium-specific bacteriophage P22

Insertion of the genes encoding the holin protein and the lysozyme can be achieved using any suitable technique, which include but are not necessarily limited to homologous recombination of one or more DNA polynucleotides that comprise one or more open reading frames which encode the described proteins. The homologous recombination can be achieved using any suitable techniques, a non-limiting example of which is provided in the Examples of this specification. In alternative embodiments, one or more guide RNA directed nucleases can be used to insert one or more DNA templates that comprises genes encoding the holin protein and the lysozyme.

Expression of the holin protein and the lysozyme can be driven using any suitable promoter that is functional in bacteria and is operably linked to the genes encoding the holin protein and the lysozyme. In embodiments, a heterologous promoter can be used. In embodiments, an endogenous promoter can be used. In embodiments, insertion of the genes encoding the holin protein and the lysozyme is configured such that expression of both genes is driven by an endogenous sseA promoter. Thus, in embodiments, the genes encoding the holin protein and the lysozyme are introduced into chromosomes of the bacteria downstream of the endogenous sseA promoter.

In certain examples which are described in more detail in the Examples of this disclosure, expression of the holin protein and the lysozyme results in intracellular autolysis.

The disclosure also provides for introducing into an animal in need thereof an effective amount of the described modified bacteria to produce a prophylactic or therapeutic effect against a bacterial infection in the individual. The modified bacteria can be introduced into the animal using any suitable route. In embodiments, the modified bacteria are introduced orally into the individual. In a non-limiting example, the modified bacteria are introduced into the animal by including the modified bacteria in drinking water. In certain examples, introducing the modified bacteria into an animal stimulates production of inflammatory cytokines in the individual. In certain embodiments, introducing the modified bacteria reduces Salmonella in the individual. In certain embodiments, reducing Salmonella comprises reducing Salmonella gut colonization, fecal shedding of Salmonella, or a combination thereof.

The animal to which the modified bacteria are introduced is not particularly limited. In embodiments, the animal is a human or a non-human animal. In some examples, the animal is an avian animal.

The disclosure also provides vaccine formulations comprising the described modified bacteria. In embodiments, the disclosure provides modified bacteria that are considered a vaccine strain.

The disclosure also provides articles of manufacture comprising the described modified bacteria. Such articles of manufacture can include a sealable container in which the modified bacteria are held. Articles of manufacture can further comprise printed material that provides instructions for administering the modified bacteria to an individual in need thereof. The disclosure also provides modified bacteria that are cryopreserved or lyophilized for use in reconstitution and in the described methods.

As described further below, non-limiting embodiments of the disclosure are illustrated using lysis genes 13 and 19 of S. typhimurium-specific bacteriophage P22 that are essential for Salmonella lysis. Gene 13 encodes holin protein, which disrupts the cell membrane by forming pores and provides the access of lysozyme, encoded by gene 19, to the bacterial cell wall for the lysis of Salmonella spp. This live Salmonella vaccine strain, namely the intracellular autolytic SE serovar Typhimurium LT2, is referred to herein as STLT2^{+P13+19}. This vaccine strains was examined by characterizing the bacterial growth, adherence activity, intracellular viability, and the mRNA expression of inflammatory cytokines and endosomal toll-like receptors (TLRs) using chicken macrophage cells as well as its in vivo effectiveness against Salmonella colonization in chicken gut through assessing the stimulation of immunity, prevention of intestinal Salmonella colonization, and gut microbial compositions.

### BRIEF DESCRIPTION OF THE FIGURES

FIG. 1A. Schematic diagram of pDS132-down-13/19-sseA construction. Eluted genes 13 and 19 with XbaI/BamHI double digestion from pET-16b-13/19 included ribosomal binding site (rbs).

FIG. 1B. Schematic diagram showing how the sseA open reading frame (orf) was inserted upstream of rbs-genes 13 and 19 and downstream of sseA orf was inserted downstream of rbs-genes 13 and 19.

FIG. 1C. Schematic diagram showing completed plasmid pDS132-down-13/19-sseA which was transformed into conjugal donor strain E. coli B2155 for conjugal DNA transfer to LT2.

FIG. 2. Comparison of bacterial growth and viability between STLT2^{+P13+19} (sse-13/19) and LT2 (WT). Growth curve (A) and viability at initiation (0-hr) and stationary phases (8-hr) (B) of STLT2^{+P13+19} and LT2. Data are expressed as mean±SD. **p<0.01 compared with WT.

FIG. 3. Adherent ability (A) and intracellular viability (B) of STLT2^{+P13+19} (INC-ATLY) and LT2 (WT) in HD11 cells. Data are expressed as mean±SD. ***, p<0.001 compared with WT.

FIG. 4. Relative mRNA expression of P22 genes (13 and 19) shown in panel (A), SPI-1 genes (invA, invE, and invF), and SPI-2 genes (pipB, sifA, sseA, sseJ, and sspH2) in STLT2^{+P13+19} (INC-ATLY) and LT2 (WT) at 24-hr post-infection in HD11 cells shown in panel (B). Data are expressed as mean±SD. ***, p<0.001 compared with WT.

FIG. 5. Relative mRNA expression of IL-1β, IL-6, IL-8, IL-10, IL-18, LITAF, iNOS, TLR3 and TLR7 genes in STLT2^{+P13+19} (INC-ATLY) or LT2 (WT)-infected HD11 cells. Data are expressed as mean±SD. **, p<0.01 and ***, p<0.001 compared with WT-infected HD11 cells.

FIG. 6. Comparison of Salmonella colonization and shedding in chicken with or without STLT2^{+P13+19} vaccination. STLT2^{+P13+19} was orally given to the chickens at days 1 and 7. The chickens were challenged with S. typhimurium or S. enteritidis at day 14. Salmonella fecal shedding (A) and intestinal including ileal (B), jejunal (C) and cecal (D) colonization were examined. Data are expressed as mean±SD. *, p<0.05 and **, p<0.01 compared with Salmonella; #, p<0.05 and ###, p<0.001 compared with S. typhimurium.

FIG. 7. Immunity stimulation in chickens vaccinated by STLT2^{+P13+19}. The relative mRNA expressions (A) and serum cytokine levels of iNOS (B), IL-6 (C), IL-8 (D), IL-12 (E), and TNF-α (F) in chickens challenged by S. typhimurium (ST) or S. enteritidis (SE) with or without STLT2^{+P13+19} vaccination (V) were measured and compared. Data are expressed as mean±SD. Different letters (a, b, and c) indicate significant differences among groups at p<0.05.

FIG. 8. Chicken gut intestinal microbiota modulation by STLT2^{+P13+19} vaccination. The influences of STLT2^{+P13+19} oral vaccination on chicken gut microbial composition were assessed at both phylum (A) and genus (B) levels. ST, chicken challenged with S. typhimurium; ST+V, chicken with S. typhimurium challenge and STLT2^{+P13+19} vaccination; SE, chicken challenged with S. enteritidis; SE+V, chicken with S. enteritidis challenge and STLT2^{+P13+19} vaccination.

FIG. 9. Diagram of homologous recombination without chloramphenicol resistance gene (cat). R6Kori, mobRP4, cat, and sacB were deleted when homologous recombination occurred.

FIG. 10. Confirmation of STLT2^{+P13+19} by PCR amplification. The amplified PCR product size was 0.9 kb for LT2 (STLT2 WT) and 1.8 kb for STLT2^{+P13+19} (STLT2 INC-ATLY), respectively.

### DETAILED DESCRIPTION

Unless specified to the contrary, it is intended that every maximum numerical limitation given throughout this description includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

As used herein and in the appended claims, the singular forms “a”, “and” and “the” include plural references unless the context clearly dictates otherwise.

The disclosure includes all polynucleotide sequences described herein, the DNA and RNA equivalents of all such polynucleotides, including but not limited to cDNA constructs, and all polypeptides encoded by the polynucleotides described herein. The disclosure includes all amino acid sequences described herein, and all functional fragments thereof. A functional fragment is a contiguous segment of a described protein that retains its ability to participate in the killing of bacteria.

In embodiments, the disclosure provides modified bacteria. The modified bacteria contain one or more genomic modifications. The genomic modifications comprise introducing one or more DNA polynucleotides into the genomes of the bacteria such that the genomes of the bacteria are altered to encode and produce a holin protein and to encode and produce a lysozyme. The holin protein and the lysozyme proteins can be from any source. In embodiments, the holing and lysozyme proteins are adapted from phage encoded proteins. In embodiments, the holin protein is encoded by gene 13 of Salmonella typhimurium-specific bacteriophage P22, and/or the lysozyme is encoded by gene 19 of the S. typhimurium-specific bacteriophage P22. In one example, the holin protein is encoded by gene 13 of Salmonella typhimurium-specific bacteriophage P22 and the lysozyme is encoded by gene 19 of the S. typhimurium-specific bacteriophage P22.

In certain examples the modified bacteria are non-pathogenic to the individual to which the modified bacteria are administered. The modified bacteria may be rendered non-pathogenic by modification, or the modified bacteria may be non-pathogenic prior to being modified. In embodiments the modified bacteria are Salmonella. In embodiments, the modified bacteria non-pathogenic Salmonella. In some embodiments, the non-pathogenic bacteria are Salmonella enterica (SE). In embodiments, the modified bacteria non-pathogenic Salmonella. In some embodiments, the non-pathogenic bacteria are Salmonella enterica (SE). In some embodiments, the SE are SE intracellular autolytic SE serovar Typhimurium (S. typhimurium). In an embodiment, the disclosure provides modified bacteria that may be considered a vaccine strain. A non-limiting embodiment of a vaccine strain demonstrated using SE serovar Typhimurium LT2 is referred to herein as STLT2^{+P13+19}. Wild type SE serovar Typhimurium LT2 are available under American Type Culture Collection (ATCC) number 19585.

The holin protein and the lysozyme proteins are not particularly limited and may be encoded by, for example, any phage genome. In certain examples, the holin protein is encoded by gene 13 of Salmonella typhimurium-specific bacteriophage P22, and/or the lysozyme is encoded by gene 19 of the S. typhimurium-specific bacteriophage P22. In one example, the holin protein is encoded by gene 13 of Salmonella typhimurium-specific bacteriophage P22 and the lysozyme is encoded by gene 19 of the S. typhimurium-specific bacteriophage P22. Representative and non-limiting examples of amino acid sequences that were used to produce the modified bacteria described in the Examples are as follows: The amino acid sequence for Salmonella bacteriophage holin encoded by P22 gene 13 is:

(SEQ ID NO: 1)
MKKMPEKHDLLTAMMAAKEQGIGAILAFAMAYLRGRYNGGAFKKTLIDA
TMCAIIAWFIRDLLVFAGLSSNLAYIASVFIGYIGTDSIGSLIKRFAAK
KAGVDDANQQ.

The amino acid sequence of endolysin (also referred to herein as lysozyme), encoded by P22 gene 19. The amino acid sequence of lysozyme is:

(SEQ ID NO: 2)
MMQISSNGITRLKREEGERLKAYSDSRGIPTIGVGHTGKVDGNSVASGM
TITAEKSSELLKEDLQWVEDAISSLVRVPLNQNQYDALCSLIFNIGKSA
FAGSTVLRQLNLKNYQAAADAFLLWKKAGKDPDILLPRRRRERALFLS.

The present disclosure also pertains to variants of the described proteins. Such variants may have an altered amino acid sequence which can function in various ways. Variants can be generated by mutagenesis, i.e., discrete point mutation or truncation, or by any other suitable approach. Biologically active portions of a protein or peptide fragment of described proteins include polypeptides comprising amino acid sequences sufficiently identical to or derived from the amino acid sequence of a protein of the disclosure, which include fewer amino acids than the full length protein and exhibit at least one activity of the corresponding full-length protein. Typically, biologically active portions comprise a domain or motif with at least one activity of the corresponding protein. In certain embodiments, the disclosure includes homologous proteins. Homology in certain embodiments is at least 50%, 65%, 75%, 80%, 85%. In certain embodiments, homology is at least 90%, 95%, 97%, 98%, or at least 99% compared to the amino acid sequences of the described holin and lysozyme proteins. Vectors and polynucleotides for use in producing the modified bacteria are provided by the disclosure. The polynucleotides may comprise any sequence that selectively hybridizes to a polynucleotide encoding one or both of the described proteins. Cells comprising the vectors are included in the disclosure. The disclosure also includes the described methods of making the modified bacteria.

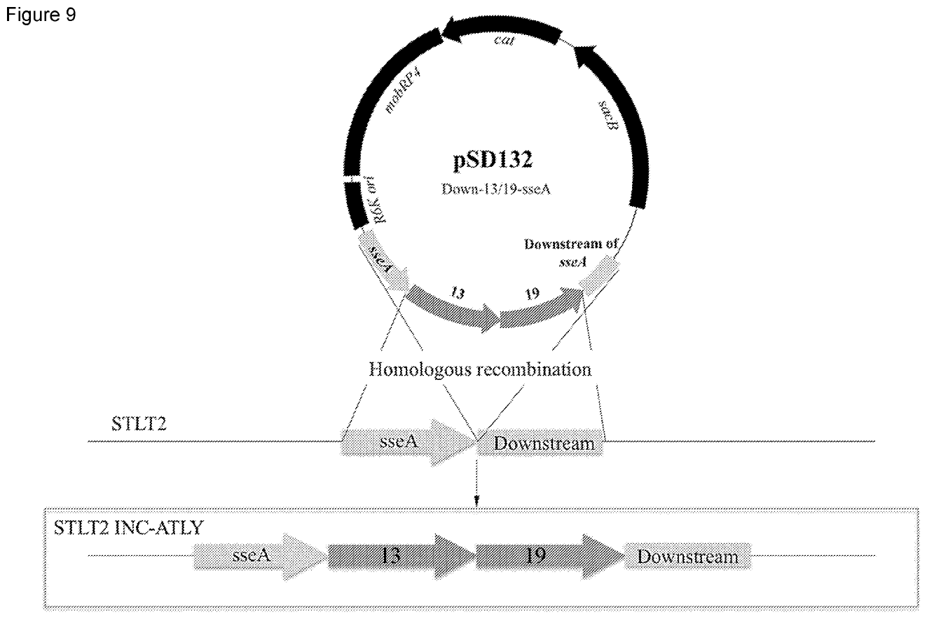

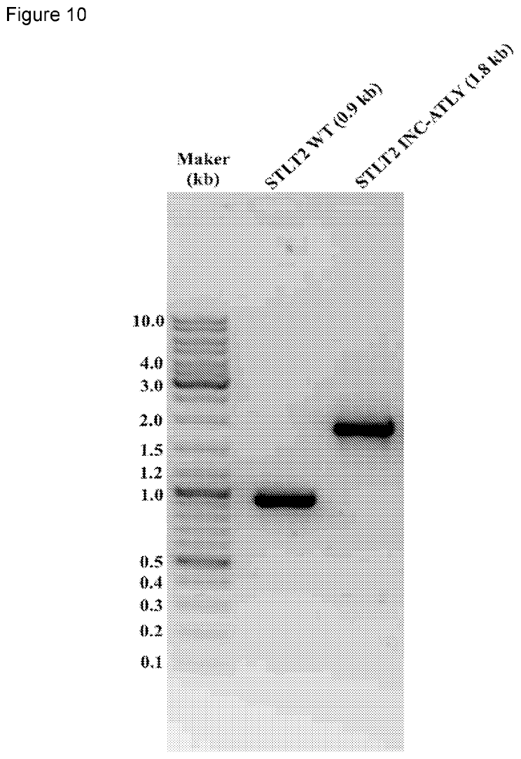

The genes encoding the holing and lysozyme can be introduced into chromosomes of bacteria using any suitable approach. One non-limiting illustration of compositions and methods used to produce the modified bacteria is shown in (FIG. 9), which includes a diagram of a homologous recombination approach. The diagram shows homologous integration into the sseA gene. Plasmid pSD132 includes left and right homology arms comprising segments of the sseA gene of sufficient length so that the plasmid-provided construct includes a segment comprising the 13 and 19 genes flanked by sseA sequences. As such, the segment of the described plasmid includes open reading frames that encode the described proteins that are recombined in a manner such that expression of the 13 and 19 genes is driven by expression from the upstream, endogenous sseA gene promoter.

In the example shown in FIG. 9, the 13 and 19 genes are recombined as shown in the sseA gene. The nucleotide sequence for the sseA gene is: GAGGGGAATGATGATAAAGAAAAAGGCTGCGTTTAGTGAATATCGTGATTTAGA GCAAAGTTACATGCAGCTAAATCACTGTCTTAAAAAATTTCACCAAATCCGGGCT AAGGTGAGTCAACAGCTTGCTGAAAGGGCAGAGAGCCCCAAAAATAGCAGAGA GACAGAGAGTATTCTTCATAACCTATTTCCACAAGGCGTTGCCGGGGTTAACCAG GAGGCCGAGAAGGATTTAAAGAAAATAGTAAGTTTGTTTAAACAACTTGAAGTA CGACTGAAACAACTTAATGCTCAAGCCCCGGTGGAGATACCGTCAGGAAAAACA AAAAGGTAA (SEQ ID NO:3)/NCBI accession NP_460362.1). When in recombined into Salmonella the sseA operon sequence is: GACTCGCTACGCTCGCCCTTCGGGCCGCCGCTAG (SEQ ID NO: 47) as described in Xu, X., & Hensel, M. (2010). Systematic analysis of the SSRAB virulon of Salmonella enterica. Infection and Immunity, 78(1), 49-58. https://doi.org/10.1128/iai.00931-09.

The sseA gene encodes the SSEA protein, the amino acid sequence of which is:

(SEQ ID NO: 4)
MMIKKKAAFSEYRDLEQSYMQLNHCLKKFHQIRAKVSQQLAERAESPKN
SRETESILHNLFPQGVAGVNQEAEKDLKKIVSLFKQLEVRLKQLNAQAP
VEIPSGKTKR/(NCBI accession NP_460362.1).

The approach described in FIG. 9 illustrates producing a strain referred to herein as SE serovar Typhimurium LT2 (STLT2^{+P13+19}), but is not meant to be limiting. The disclosure includes integration of the 13 and 19 genes as a bi-cistronic element, or integration of each gene separately, provided the described proteins are operably linked to a suitable promoter that is functional in the modified bacteria. Thus, insertion of the described genes can be achieved using, for example, guide-directed RNA nucleases, TALONS, zinc fingers, and other designer nucleases that will be apparent to those skilled in the art. The disclosure includes introducing the described genes in the N-C terminal order as shown in FIG. 9, and also reversing the order so that the 19 gene is N-terminal to the 13 gene.

The sseA gene is used as a homologous recombination site so that the sseA promoter drives expression of the genes, but is a non-limiting embodiment. In alternative embodiments, the promoter that is used so that it is operably linked to and therefore drives expression of the described genes can be heterologous to the bacteria, meaning it is taken or derived from a different organism, or it may be endogenous to the organism, as illustrated using the sseA construct. The disclosure also includes introducing a promoter that is endogenous to the organism but has been introduced into a new location such that it can drive expression of the described genes, but in a different locus than the endogenous locus.

Expression of the two described proteins may be due to translation from a single mRNA. Translation may be discontinuous such that the proteins are produced as separate proteins. As such, the disclosure includes separating the open reading frames encoding proteins by, for example, an internal ribosome entry site (IRES), or a ribosomal skipping peptide, many of which are known in the art and can be adapted for use in the present disclosure. In embodiments used in the Examples below, translation occurs on a single mRNA strand containing the sequences for both genes 13 and 19. These are oriented next to each other in unidirectional tandem open reading frames (ORF). Accordingly, the gene 19 is located directly downstream of gene 13, with the stop codon of gene 13 ORF overlapping with the start codon of the gene 19 ORF. These overlapping ORFs on P22 genes 13 and 19 are configured such that the mRNA forms a stem-loop structure. This stem-loop structure in unidirectional tandem ORFs cause translational coupling, which is a form of ribosome re-initiation that enables the beginning of translation for a second downstream protein that is nearby, which is in this example is the protein coded by gene 19. (See, for example, Rennell, D., & Poteete, A. R. (1989). Genetic analysis of bacteriophage P22 lysozyme structure. Genetics, 123(3), 431-440, and Wright, B. W., Molloy, M. P., & Jaschke, P. R. (2021). Overlapping genes in natural and engineered genomes. Nature Reviews Genetics, 23(3), 154-168.

The disclosure comprises isolated modified bacteria, cell cultures comprising the modified bacteria, and kits comprising the modified bacteria. In an embodiment, a kit comprises one or more sealed containers comprising the modified bacteria, and may include printed material that provides instructions for use of the modified bacteria.

The disclosure includes media in which the bacteria are cultured. The disclosure includes the modified bacteria held in any form of container, e.g., a vessel, containing the bacteria, including the vessels in all stages of operation. In embodiments, the vessels are cell culture dishes, flasks, sealable tubes, or bioreactors. In embodiments, the bioreactors have a volume of a least 2 liters. In embodiments, a bioreactor used to create and/or produce modified bacteria of this disclosure has a volume that is from 2 to up to 3 million liters, inclusive, and include all numbers and ranges of numbers there between.

The modified bacteria may be used without other components, or with any suitable other agent, such as a diluent, such as by placing the modified bacteria into an aqueous solution, including but not necessarily limited to drinking water. Compositions comprising the modified bacteria of the disclosure may also include other antimicrobial agents. For example, the modified bacteria can be used with antibiotics that are members of classes such as aminoglycosides, beta lactams (with or without beta lactamase inhibitor such as clavulanic acid), macrolides, glycopeptides, polypeptides, cephalosporins, lincosamides, ketolides, rifampicin, polyketides, carbapenem, pleuromutilin, quinolones, streptogramins, oxazolidinones, or lipopeptides.

In embodiments, the disclosure includes administering the described modified bacteria to a human individual in need thereof. In other embodiments, the disclosure includes veterinary approaches, and thus in this aspect pertains to use of the described modified bacteria in non-human mammals. In embodiments, the modified bacteria are administered to avian animals. In embodiments, the avian animals are any type of poultry. In embodiments, the avian animals are Galliformes and thus include any members of the order of heavy-bodied ground-feeding birds that includes turkey, grouse, chicken, New World quail and Old World quail. In embodiments, the avian animals are domesticated fowl, including but not limited to domesticated chickens and turkeys. In embodiments, the chickens are Gallus gallus, such as Gallus gallus domesticus. In embodiments, the chickens are roosters or hens. In embodiments, the chickens are broiler chickens. In embodiments, the avian animals are adults or juveniles. In embodiments, vaccines of this disclosure may be administered to a population of avian animals, i.e., a flock. In embodiments, from 50-85% or more members of the flock are vaccinated to achieve, for example, herd or flock immunity.

In other non-limited embodiments, the described bacteria are administered to a ruminant, including but not necessarily limited to bovines, sheep, antelopes, deer, giraffes, and their relatives, and further can include pseudoruminants, such as the camelids. In embodiments, the ruminant is bovine mammal that is a member of the genus Bos, such as oxen, cows, and buffalo. In one embodiment the ruminant is a dairy cow.

In an embodiment, the disclosure includes administering modified bacteria of this disclosure to any member of the genus Sus, and therefore encompasses practicing the invention with any swine, examples of which are not limited to the domestic pig (i.e., Sus domesticus), also commonly referred to as a swine or a hog.

The disclosure also includes administering the vaccines to non-bovine and non-ruminant mammals, including but not necessarily limited to equines, canines, and felines.

Compositions comprising the described modified bacteria can be directed to the mucosal lining, where, in residence, they kill colonizing disease bacteria. The mucosal lining includes, for example, the upper and lower respiratory tract, eye, buccal cavity, nose, rectum, vagina, periodontal pocket, intestines and colon. Due to natural eliminating or cleansing mechanisms of mucosal tissues, the disclosure includes addition of agents that exhibit adhesion to mucosal tissues.

For administration of the described modified bacteria, a therapeutically effective dose can be estimated initially either in cell culture assays or in animal models. The animal model may also be used to achieve a desirable concentration range and route of administration. Such information can then be used to determine useful doses and routes for administration in humans non-human animals. The exact dosage is chosen by the individual physician or other health care provider such as an individual skilled in veterinary medicine in view of the individual to be treated. Dosage and administration can be adjusted to provide sufficient amounts of the modified bacteria to maintain the desired effect. Additional factors which may be taken into account include the type and severity of the disease state, age, weight of the subject; diet, desired duration of treatment, method of administration, and time and frequency of administration.

The disclosure includes methods of treating bacterial infections, related infections or conditions, including antibiotic-resistant bacteria, including wherein the bacteria or a subject infected by or exposed to the particular bacteria, or suspected of being exposed or at risk, is administered an amount modified bacteria of the disclosure that is effective to kill the particular bacteria. It is expected that methods of the present disclosure will be applicable to any animal that is susceptible Salmonella infection, or is otherwise in need of or would benefit from receiving a composition of this disclosure. In certain approaches the modified bacteria are used to kill or reduce the growth of pathogenic bacteria. In embodiments, the bacteria that are affected by the modified bacteria comprise any type of pathogenic Salmonella, S. aureus, P. aeruginosa, or E. coli. In certain embodiments, use of the modified bacteria of the disclosure results in eradication of a bacterial population from an infection, such as from an infection of an organ, tissue, skin, biological fluid from an individual, and/or reduces or eliminates fecal shedding of bacteria. Any result produced using the modified bacteria can be compared to a suitable control. In embodiments, use of the modified bacteria provides an improved result relative to the control. In embodiments, a control comprises bacteria that are unmodified, or a vaccine formulation that has been previously described.

In embodiments, the modified bacteria are used for killing, decolonization, prophylaxis or treatment of Gram-positive or Gram-negative bacteria, including bacterial infections or related conditions. In embodiments, use of the modified bacteria reduce and/or eradicate bacterial persister cells and/or dormant viable but non-culturable (VBNC) bacteria.

In embodiments, use of the modified bacteria produces a humoral immune response, a cell mediated immune response, or a combination thereof. In embodiments, use of the described modified bacteria stimulates production of cytokines, as more fully described in the Examples. In embodiments, use of the modified bacteria stimulates expression of Toll-like receptors, as more fully described in the Examples. In embodiments, use of the modified bacteria stimulates expression of interleukins, as more fully described in the Examples.

The disclosure may be better understood by reference to the following non-limiting Examples, which are provided as exemplary of the disclosure.

### Example 1

This Example provides the material and methods used to produce the results discussed in the following Examples.

Bacteria strains and culture condition: Three SE strains non-pathogenic Typhimurium ATCC 19585 (LT2), pathogenic Typhimurium ATCC 14028 (STY), and pathogenic Enteritidis ATCC 13076 (SEE) were purchased from American Type Culture Collection (ATCC, Manassas, VA, USA). E. coli TOP10 was purchased from Invitrogen (Carlsbad, CA, USA) and E. coli DH5α and B2155 were obtained from Delaware University, Newark, DE, USA. All bacteria strains were cultivated overnight in Luria-Bertani (LB) broth (Difco, Becton, Dickinson and Co., Sparks, MD, USA) at 37° C. Diaminopimelic acid (DAP, 0.3 mM) was used for the growth of DAP auxotroph, E. coli B2155. Ampicillin (100 μg/mL) was used for culturing strains containing pCR2.1 or pET16b derivative plasmids. Chloramphenicol (30 μg/mL) was used for culturing strains containing pDS132 derivative plasmids.

Cell line and culture condition: HD-11 (Chicken macrophage) cells were obtained from the Immunobiology Branch of the Food and Drug Administration (Laurel, MD, USA). The HD-11 cells were cultured in Dulbecco's modified Eagle's medium (DMEM, Corning, Manassas, VA, USA) containing 10% fetal bovine serum (FBS; Corning, Manassas, VA, USA) with 5% CO₂ and 100% humidity at 37° C. in 150 cm² T-flask (Corning, Manassas, VA, USA). The HD-11 cells were cultured in 24-well plates overnight for harvesting monolayer.

Construction of autolytic Salmonella vaccine strain: The genomic DNA of LT2 and bacteriophage P22 (ATCC, Manassas, VA, USA) was isolated using DNeasy Blood & Tissue Kit (Qiagen, Germantown, MD, USA). The 753-bp genes 13 and 19 fragments from bacteriophage P22 were amplified with PCR using primers concatenated with NdeI and BamHI linkers. The 327-bp sseA (SPI-2 gene) and 317-bp downstream of sseA fragment from LT2 were amplified with PCR using primers concatenated with XbaI & XhoI XbaI linkers and BamHI SacI linkers, respectively. PCR was performed in Mastercycler (Eppendorf, Hauppauge, NY, USA), and programmed for 1 cycle of 94° C. for 2 min, 30 cycles of 94° C. for 30 sec, 60° C. for 30 sec, 72° C. for 60 sec, and 1 cycle of 72° C. for 2 min.

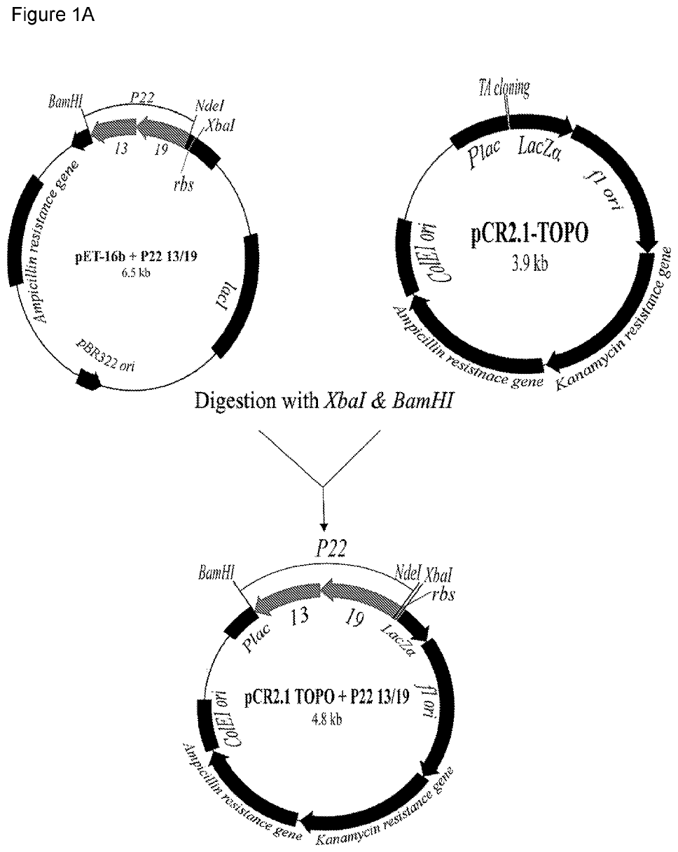

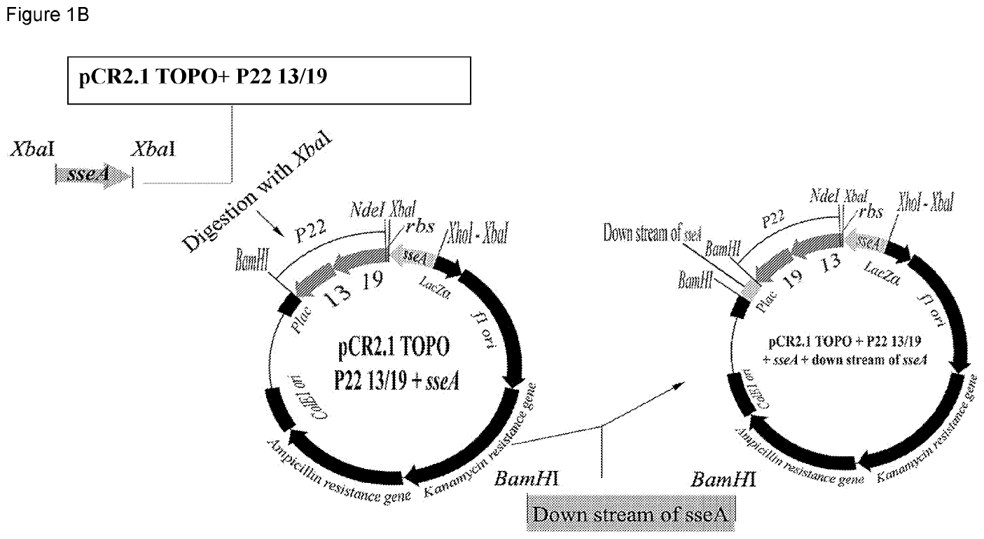

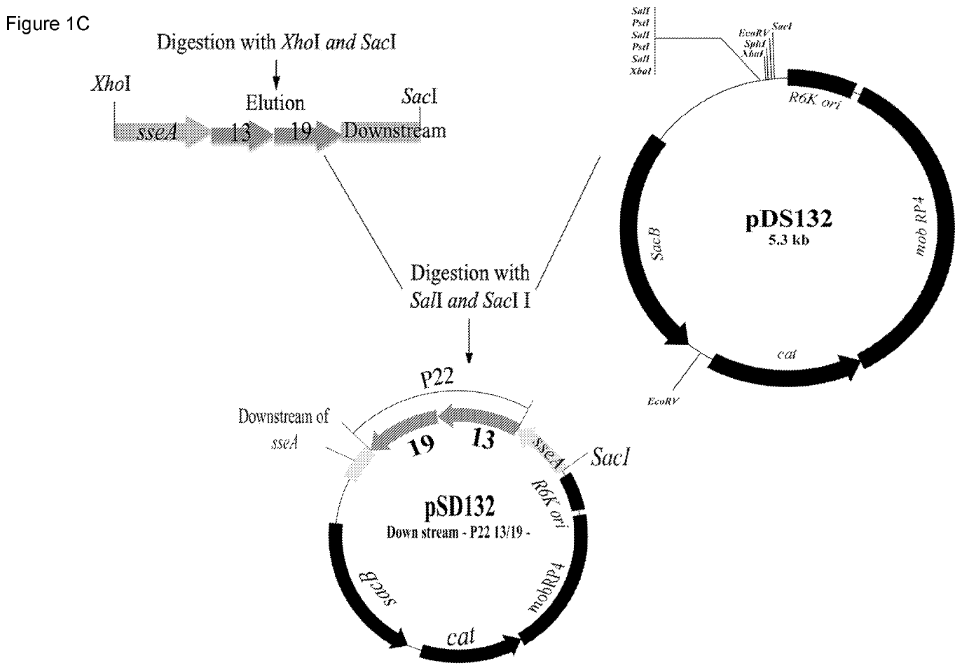

The 13/19 gene amplicon was double-digested with NdeI BamHI and ligated into NdeI BamHI double-digested pET16b, forming pET16b-13/19 for further transformation and plasmid isolation. Then, the transformation of pET16b-13/19 into E. coli TOP10 was performed by heat-shock transformation with chemically competent cells and followed by selection on LB agar with 100 μg/ml ampicillin. The pET16b-13/19 was isolated using Plasmid DNA purification kit (iNtRON Biotechnology, Lynnwood, WA, USA). The isolated pET16b-13/19 was double-digested with XbaI BamHI to elute ribosomal binding site (rbs) and genes 13 and 19 to be ligated into XbaI BamHI double-digested pCR2.1, forming pCR2.1-13/19 for further transformation into E. coli TOP10 and plasmid isolation as described above. The sseA amplicon was digested with XbaI and ligated into XbaI-digested pCR2.1-13/19, forming pCR2.1-13/19-sseA for further transformation into E. coli TOP10 and plasmid isolation as described above. The sseA downstream amplicon was digested with BamHI SacI and ligated into double-digested pCR2.1-13/19-sseA, forming pCR2.1-down-13/19-sseA for further transformation into E. coli TOP10 and plasmid isolation as described above. The isolated pCR2.1-down-13/19-sseA was double-digested with XhoI/SacI to elute sseA-rbs-13/19-sseA downstream fragment and ligated into SalI/SacI-double digested pDS132, forming pDS132-SR13/19SD for further transformation into E. coli DH5α and plasmid isolation as described above. The isolated pDS132-down-13/19-sseA was transformed into E. coli B2155 and selected on LB agar with 30 μg/ml chloramphenicol and 0.3 mM DAP. A schematic diagram of construction of pDS132-down-13/19-sseA is shown in FIG. 1.

E. coli B2155-pDS132-down-13/19-sseA (donor) was grown in LB broth with g/ml chloramphenicol and 0.3 mM DAP, and LT2 (recipient) was grown in LB broth overnight. Then cultures of the donor and recipient were mixed at the ratio of 1:1. Mixed bacteria cells were centrifuged at 13,000 rpm for 1 min, and the bacteria pellet was resuspended in 0.1 ml LB broth with 0.3 mM DAP. The suspended bacteria mixture was dropped on LB agar with 0.3 mM DAP and incubated overnight at 37° C. for conjugal DNA transfer. The overnight culture of mixed bacteria cells was spread on LB agar containing 30 g/ml chloramphenicol and grown overnight at 37° C. to eradicate DAP auxotroph E. coli B2155 and non-conjugant LT2. Subsequently, the cultures were transferred to LB broth containing 30 μg/ml chloramphenicol and grown overnight at 37° C., and then transfer to LB broth without chloramphenicol and grown overnight at 37° C. for the integration of genes 13 and 19 into genomic DNA of LT2, followed by selection on LB agar with 5% sucrose. The integration was determined by PCR amplification with sseA orf upstream specific primer and 317 bp sseA gene downstream R primer.

Growth, adherence activity, and intracellular viability analyses of STLT2^{+P13+19}: STLT2^{+P13+19} and LT2 were grown overnight in 3 ml LB broth. Concentration of overnight-cultured bacteria strains was adjusted to 0.01 at OD_{600 nm} in 50 ml LB broth and incubated at 37° C., 180 rpm. Bacterial growth was measured at OD_{600 nm} every 2 hr for a 10-hr duration. Viability of STLT2^{+P13+19} and LT2 was determined by colony forming unit (CFU) assay at 0 and 8 hr of culture. The adherent and intracellular STLT2^{+P13+19} or LT2 were measured and compared in accordance with the previous method by Peng et al. (2015).

Animal experiments: One-day-old specific-pathogen-free chicks were purchased from Charles River (Frederick, MD) and housed at 37° C., 12 h/12 h light cycle, and grown with free access of feed and water, following guidelines and the protocol approved by the Institutional Animal Care and Use Committee (IACUC, protocol number 1160542-2) at the University of Maryland. The chickens were further divided into A, B, C, and D groups (n=15/group).

An aliquot 100 μL of overnight culture of STLT2^{+P13+19} suspended in PBS was fed to chicken (Group B & D: 10⁹ CFU/chick) with oral gavage at day 1 and day 8. At day 15, 100 μL of STY (Group A & B) or SE (Group C & D) suspension containing 10⁹ CFU bacteria was fed to each chick through oral gavage. The chicks were reared thereafter for 2 weeks, given regular tap water and fed with chicken starter diet (mash) without growth promoters or synthetic chemicals. At the end of second, third, and fourth week, 5, 5, and 5 chicks from each group were euthanized.

Starting from week 1, 15 fecal samples from each group were collected. Cecum, jejunum, and ileum from euthanized chicken were separated and collected. The number of Salmonella in the fecal and intestinal samples was quantified by plating on Xylose Lysine Deoxycholate (XLD) agar, while the number of STY or SE was quantified by plating on XLD agar with 100 μg/ml kanamycin. At the end of second, third, and fourth week, blood was collected from the heart of the euthanized chicken for further cytokine analyses.

Enzyme-Linked Immuno-Sorbant Assay (ELISA): Chicken whole blood samples were allowed to clot by leaving at room temperature for 30 min with no disturb. Then, the clot was removed by centrifuging at 3,000 rpm for 10 min. Supernatant was collected as designated serum and maintained at 4° C. The serum secretion levels of IL-6, IL-8, IL-12, iNOS, and TNF-α were measured using ELISA kits (MyBioSource, Inc., San Diego, CA, USA) for IL-6, IL-8, IL-12, iNOS, and TNF-α, respectively, following the protocols from the manufacture.

Quantitative PCR (qPCR): Bacterial cells, HD11 cells, or chicken whole blood was washed 3 times with 1×PBS. The RNA extraction, cDNA reverse transcription, and qPCR were performed in accordance with the previous methods by Peng et al. (2019). The 50s ribosomal protein L5 was used as the control gene for genes 13/19 and SPI-1/2 for data normalization, and glyceraldehyde 3-phosphate dehydrogenase (GAPDH) was the control for cytokines and TLRs. The oligonucleotide primers (Eurofins MWG Operon, Huntsville, AL, USA) are listed in Table 2.

Chicken gut microbial composition analysis: Microbial genomic DNA was extracted from individual chicken cecal fluid samples collected at the last day of the animal experiment using the PureLink Microbiome DNA Purification Kit (Invitrogen, Carlsbad, CA, USA) following the instructions from the manufacturer. DNA library was prepared using Nextera DNA Library Preparation Kit and Nextera Index Kit (Illumina, San Diego, CA, USA) targeting the V3-V4 regions of microbial 16S rRNA gene. Subsequently, 2×300 bp paired-end reads sequencing was performed based on Illumina MiSeq system using v3 600-cycle kit (Illumina, San Diego, CA, USA). The raw sequence data were pre-processed and quality-controlled by using BCL2FastQ, DeconSeq, and Mothur software. The 16S rRNA gene sequence variants at 97% identity were used to define the operational taxonomic units (OTUs). The relative taxonomic abundance of a specific phylum or genus was calculated through dividing the number of reads in the specific taxon by the number of reads in total 16S rRNA gene. The ANCOM package in R software was used to determine the significant difference among cecal microbial compositions in chicken from different groups.

Statistical analysis: All data were conducted with at least three times individual and independent experiments. Difference among groups was considered as significant when the P value was less than 0.05.

### Example 2

Growth of STLT2^{+P13+19} in vitro. We compared the growth and survival ability of STLT2^{+P13+19} after insertion of genes 13 and 19 into the wild-type LT2 (FIG. 1). STLT2^{+P13+19} tended to grow significantly faster than LT2, specifically at the first 10 hr. Both strains entered log phase within 2 hr of culture and reached the stationary phase after 8 hr (FIG. 1A). Specifically, LT2 was 5.34×10⁶ CFU/ml and 5.65×10⁸ CFU/ml at the initiation (2 hr) and stationary (8 hr) phases, respectively, in comparison with 5.01×10⁶ CFU/ml and 6.01×10⁸ CFU/ml for STLT2^{+P13+19} (FIG. 1), indicating that the Salmonella vaccine strain STLT2^{+P13+19} can grow well and even better than the wild type.

### Example 3

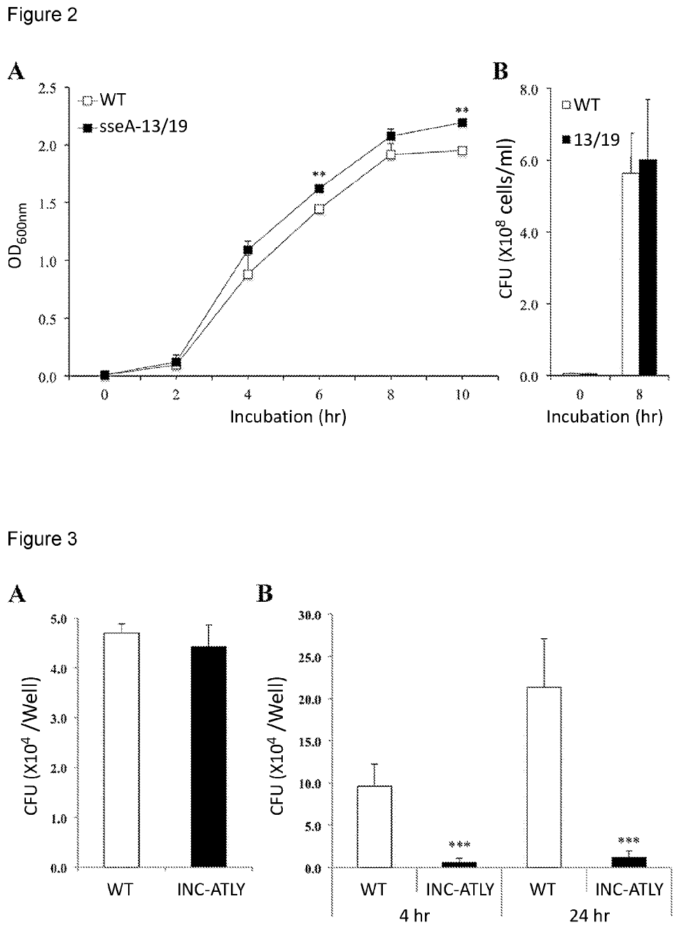

Reduced intracellular viability of STLT2^{+P13+19} in HD11 cells. The adherence ability of both STLT2^{+P13+19} and LT2 to HD11 cells and their intracellular survivability in HD11 cells were compared (FIG. 2). The adhesion abilities of both LT2 and STLT2^{+P13+19} to HD-11 cells were observed similar Adhesion however, the intracellular viability of STLT2^{+P13+19} was significantly reduced 93.59% and 94.42% LT2 at 4-hr and 24-hr post infections, respectively, compared wild-type strain LT2 (FIG. 2A) On the other hand, in terms of survival ability and multiplication, LT2 increased significantly by 2.22-fold at 24-hr post-infection compared to 4-hr post-infection, whereas STLT2^{+P13+19} merely insignificantly increased by 1.93-fold (FIG. 2B).

### Example 4

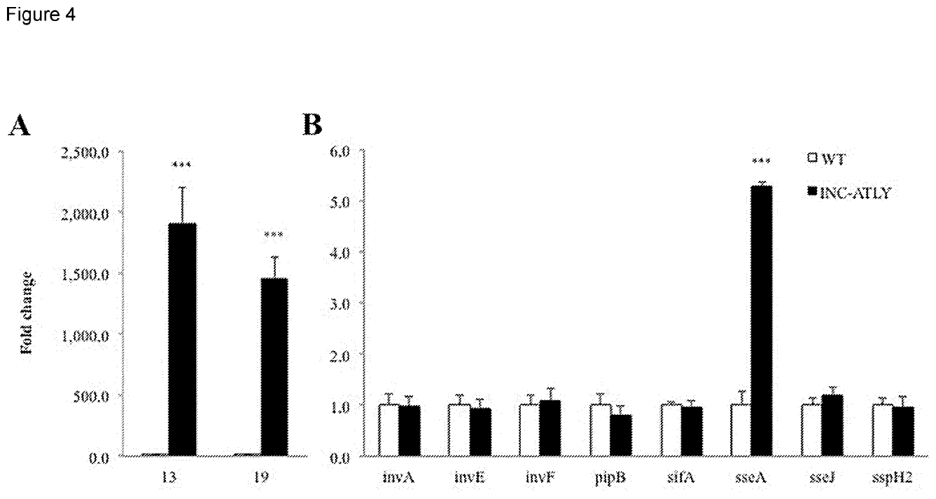

Expression of P22 holin/lysozyme genes and SPI-1/2 genes in STLT2^{+P13+19} after infecting HD11 cells. In order to confirm the successful integration of holin gene (gene 13) and lysozyme gene (gene 19) of bacteriophage P22 into the Salmonella vaccine strain, we examined their mRNA expressions at 24-hr post-infection into HD11 cells, and the results are shown in FIG. 4A. As expected, both the mRNAs were highly expressed; we observed a significantly elevated gene expression of genes 13 (˜2000 folds increasing) and 19 (˜1500 folds increasing). Furthermore, we also compared the mRNA expression of invasion- and intracellular survival-relevant genes between STLT2^{+P13+19} and LT2 after infection into HD11 cells (FIG. 4B). No significant difference was observed in SPI-1 gene expression, including invA, invE, and invF genes, and SPI-2 gene expression, including pipB, sifA, sseJ, and sspH2 genes between these two strains at 24-hr post-infection into HD11 cells. However, the mRNA expression of sseA gene in STLT2^{+P13+19} was 5-fold higher than that in LT2 after being infected into HD11 cells (FIG. 4B). Such findings demonstrate the successful integration of P22 holin and lysozyme genes into the Salmonella vaccine strain as well as the activated expression of sseA gene in the vaccine strain under intracellular condition.

### Example 5

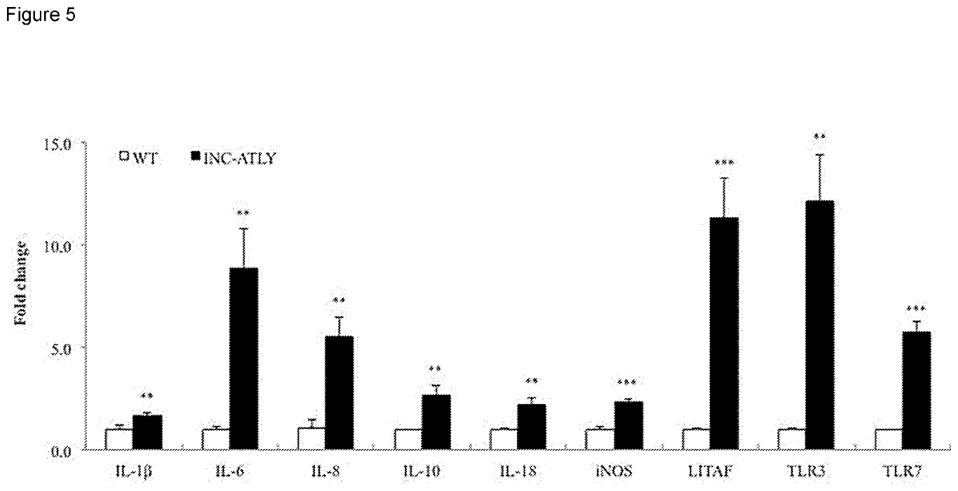

STLT2^{+P13+19}-infected HD11 cells stimulate inflammatory cytokine and endosomal TLR gene expressions. To further assess the stimulatory effect of STLT2^{+P13+19} infection on cellular innate immunity in HD11 cells, we next examine the gene expression of inflammatory cytokines and endosomal TLRs in HD11 cells infected with STLT2^{+P13+19} or LT2 (FIG. 5). For inflammatory cytokine genes, we observed that the HD11 cells with 24-hr STLT2^{+P13+19} infection were associated with 1.68, 8.85, 5.56, 2.65, 2.19, 2.36, and 11.34 folds higher mRNA expressions of IL-10, IL-6, IL-8, IL-10, IL-18, iNOS, and LITAF, respectively, in comparison with those in the HD11 cells with 24-hr LT2 infection. Meanwhile, The mRNA expression levels of endosomal TLR3 and TLR7 in STLT2^{+P13+19}-infected HD11 cells were identified to be significantly higher by 12.16 and 5.70 folds than those found in LT2-infected HD11 cells (FIG. 5), respectively.

### Example 6

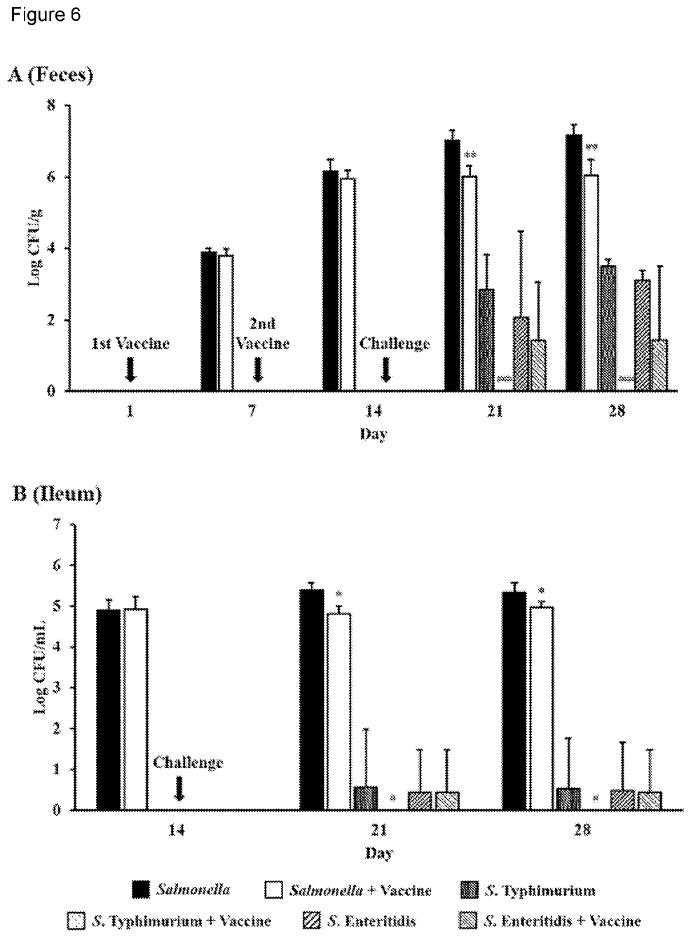

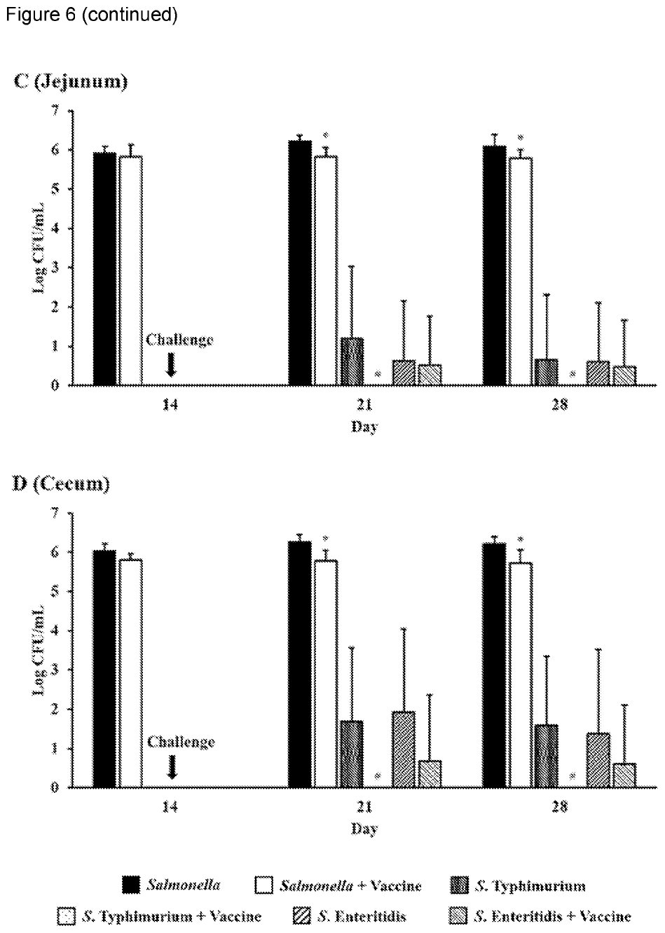

STLT2^{+P13+19} vaccination reduce Salmonella colonization in chicken gut. STLT2^{+P13+19} was orally provided to half of the chickens twice, as an initial vaccination at day 1 and another boost at day 7, followed by STY or SEE challenge in all the chickens at day 14. As shown in FIG. 6A, based on the microbiological analysis of the weekly collected chicken fecal samples, we observed that the number of Salmonella in feces was slightly (p>0.05) lowered in the chickens with STLT2^{+P13+19} vaccination at days 7 (0.079 log) and 14 (0.213 log); the difference in fecal Salmonella loads became significant (p<0.01) between the chickens with or without STLT2^{+P13+19} vaccination after 2 weeks, which exhibited 1.023 and 1.136 logs at days 21 and 28, respectively. STY and SEE were detected after the challenge at days 21 and 28. None of the fecal samples from the chickens with STLT2^{+P13+19} vaccination contained any STY, in comparison with 2.847 and 3.512 logs at days 21 and 28, respectively, in those that received no vaccine (p<0.001). However, the difference of fecal SEE loads between the chickens with or without STLT2^{+P13+19} vaccination was only numerical (p>0.05), exhibiting 0.648 and 1.651 logs less bacterial counts in the vaccine chickens at days 21 and 28, respectively.

The gut Salmonella colonization was microbiologically evaluated in a similar way at various intestinal locations including ileum (FIG. 6B), jejunum (FIG. 6C), and cecum (FIG. 6D). At day 14, the Salmonella colonization loads in the chickens with STLT2^{+P13+19} vaccination were all slightly (p>0.05) decreased, by 0.007, 0.105, and 0.234 log in ileum, jejunum, and cecum, respectively. The difference of gut Salmonella colonization became larger at days 21 and 28; the chickens that received the vaccine were associated with >0.3 log significantly less Salmonella in their ileum, jejunum, and cecum (p<0.05). Meanwhile, following the same trend as in fecal samples, no intestinal STY was detected in ileum, jejunum, and cecum of the chickens received STLT2^{+P13+19} vaccination, showing significant (p<0.05) reduction at both days 21 and 28, in comparison with those received no vaccine. However, intestinal SEE colonization was merely reduced numerically (p>0.05) by STLT2^{+P13+19}.

### Example 7

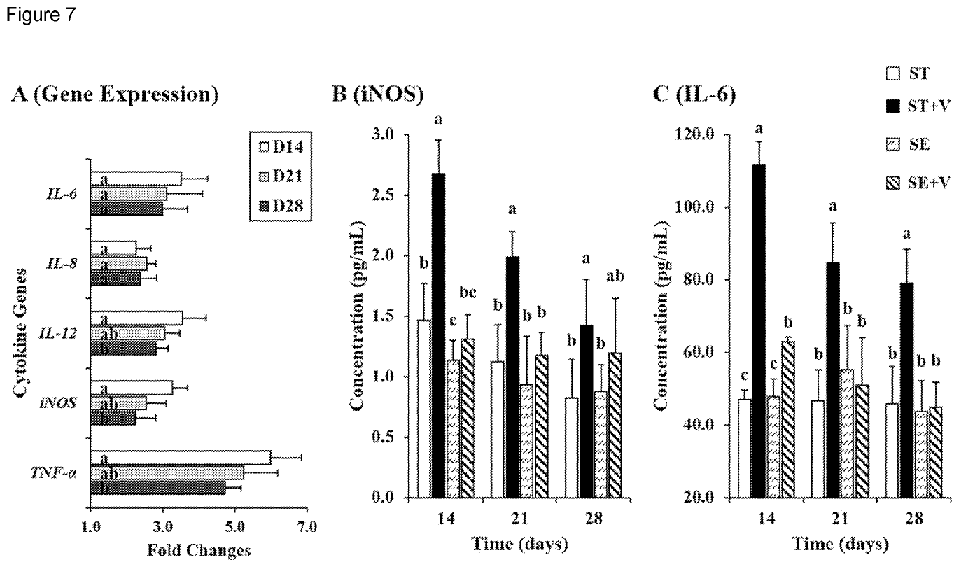

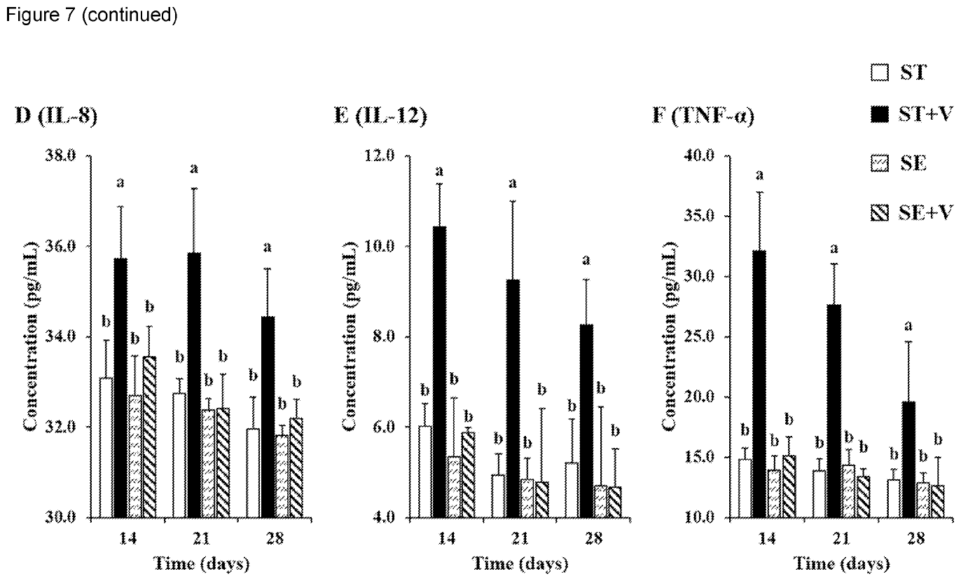

Stimulation of cytokine secretion by STLT2^{+P13+19} vaccination. We further examined the serum cytokine secretion in chickens following STLT2^{+P13+19} vaccination at transcriptional level through qPCR analysis. The gene expressions of all inflammatory cytokines examined were significantly (p<0.001) upregulated by various folds in the serum of the chickens immunized with STLT2^{+P13+19}, in comparison with those received no vaccine but challenged with STY (FIG. 7A). To be specific, STLT2^{+P13+19} promoted the mRNA expression of iNOS by 3.256 folds at day 14, 2.543 folds at day 21, and 2.234 folds at day 28. Similarly, the mRNA expressions of IL-6, IL-12, and TNF-α following STLT2^{+P13+19} vaccination were upregulated the most at day 14, exhibiting 3.492, 3.543, and 5.993 folds increasing, respectively; while the stimulatory trends of these three cytokines were compromised after 14 days, ended with 2.983, 2.808, and 4.727 folds increasing at day 28, respectively. On the contrary, the gene expression of IL-8 displayed a different trend of alteration, which was upregulated the most by 2.543 folds at day 21.

To further verify the stimulatory trends of serum cytokines, we evaluated the promotive effects of STLT2^{+P13+19} on each above-mentioned cytokine secretion individually through ELISA. Overall, the results from ELISA were in consistent with the findings based on transcriptional level (FIG. 7B-F). The serum concentration of iNOS was significantly (p<0.001) stimulated by 1.825, 1.774, and 1.731 folds by STLT2^{+P13+19} vaccination in the chickens challenged with STY, at days 14, 21, and 28, respectively (FIG. 7B); IL-6 was stimulated by 2.377, 1.821, and 1.725 folds (FIG. 7C); IL-8 by 1.080, 1.095, and 1.078 folds (FIG. 7D); IL-12 by 1.733, 1.877, and 1.588 folds (FIG. 7E); TNF-α by 2.169, 1.997, and 1.497 folds (FIG. 7F).

### Example 8

Influences of STLT2^{+P13+19} vaccination on chicken gut microbial composition. The gut intestinal microbiota was analyzed through 16S rDNA sequencing. A total of 2,082,356 raw reads were obtained from metagenomic sequencing, providing ˜173,530 reads per gut microbial sample. Based on Greengenes Database, we taxonomically classified the filtered high-quality reads and analyzed at both phylum and genus levels.

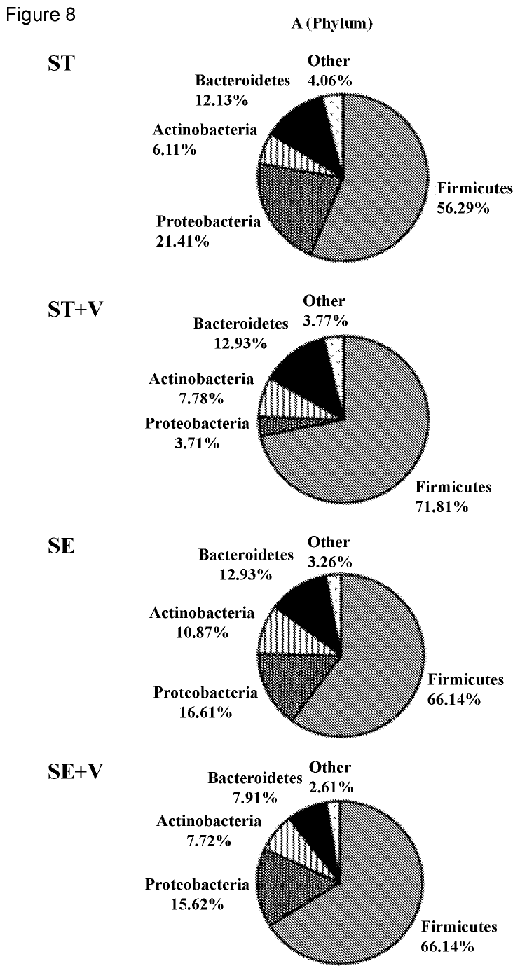

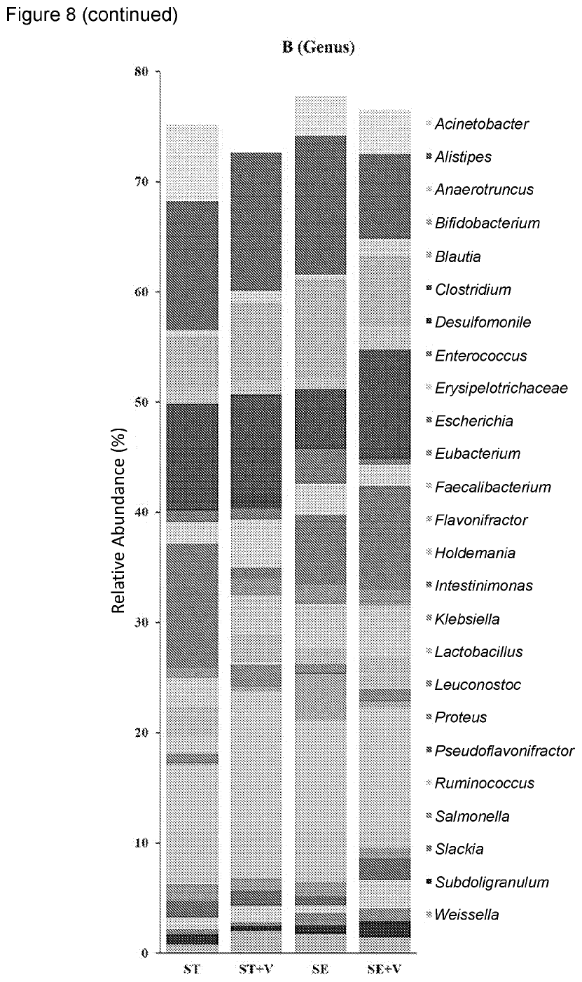

At the phylum level, Firmicutes, Bacteroidetes, Actinobacteria, and Proteobacteria were four of the most abundant phyla present in all the gut intestinal fluid samples, accounting for more than 95% of the entire bacterial abundance (FIG. 8A). The bacterial challenge led to a rapid gut colonization of STY, inducing a relatively highly abundant Proteobacteria of 21.41% at day 28; the 4-week relative intestinal abundances of Firmicutes, Bacteroidetes, Actinobacteria in STY-challenged chickens were 56.29%, 12.13%, and 6.11%, respectively. In a similar way, SEE challenge led to 4-week relative intestinal abundances of Firmicutes, Bacteroidetes, Actinobacteria, and Proteobacteria at 56.32%, 12.93%, 10.87%, and 16.61%, respectively. In comparison with the STY-challenged chickens received no vaccine, those immunized with STLT2^{+P13+19} exhibited significantly (p<0.001) lower abundance of Proteobacteria (17.70% less) and higher abundance of Firmicutes (15.52% more), accompanied with numerically (p>0.05) higher abundances of Bacteroidetes (0.80% more) and Actinobacteria (1.67% more). However, STLT2^{+P13+19} vaccination only induced numerical (p>0.05) reduction of Proteobacteria (0.99% less), while the abundances of Firmicutes, Bacteroidetes, and Actinobacteria were increased by 9.81% (p<0.01), 5.02% (p<0.05), and 3.15% (p<0.05), respectively in SEE-challenged chickens.

At the genus level, STLT2^{+P13+19} vaccination consistently expanded the relative abundances of Anaerotruncus (0.58-1.14% more), Faecalibacterium (0.59-0.96% more), Intestinimonas (0.25-1.18% more), and Ruminococcus (0.42-1.80% more), while reduced the abundance of Salmonella (0.02-0.04%0 less). Meanwhile, in comparison with the gut genera in STY-challenged chickens with no vaccine protection, those in STLT2^{+P13+19}-immunized chickens were associated with significantly lower abundances of Acinetobacter (6.87% less), Escherichia (10.35% less), Holdemania (1.41% less), and Proteus (1.10% less), as well as significantly higher abundances of Bifidobacterium (2.2700 more), Desulfomonile (1.170% more), Erysipelotrichaceae (2.4400 more), Lactobacillus (6.130% more), and Weissella (1.22% more).

TABLE 1
Bacterial Strains and plasmids used in this disclosure

| Strains and plasmids | Relevant characteristics |
|---|---|
| Bacterial Strains | |
| S. enterica serovar Typhimurium | |
| LT2 | Wild type, ATCC19585 |
| STLT2^{+P13+19} | Double cross over mutant, inserted genes 13 and 19 of |
| | bacteriophage P22 |
| E. coli | |
| DH5α | Cloning host strain |
| TOP10 | Cloning host strain |
| B2155 | Donor of conjugal DNA transfer, DAP auxotroph |
| Bacteriophage | |
| P22 | ATCC97540 |
| Plasmids | |
| pET16b | Amp^{r}, lacI, T7 promoter |
| pET16b-13/19 | pET16b derivative, containing genes 13 and 19 from P22 |
| pCR2.1 | Amp^{r}, Kan^{r}, |
| pCR2.1 13/19 | pCR2.1 derivative, containing genes 13 and 19 genes from |
| | pET16b-13/19 |
| pCR2.1 13/19-sseA | pCR2.1 13/19 derivative, containing sseA gene from LT2 |
| pCR2.1 down-13/19-sseA | pCR2.1 13/19-sseA derivative, containing down stream of sseA |
| | from LT2 |
| pDS132 | Cm^{r}, sacB, mobRP4, R6K ori |
| pDS132 down-13/19-sseA | pDS132 derivative, containing down stream of sseA, 13/19 and |
| | sseA from pCR2.1 down-13/19-sseA |

TABLE 2
Oligonucleotides and primers used for qPCR

| Organism | Target | Primer | Sequence (5′-3′) | SEQ ID NO: |
|---|---|---|---|---|
| Salmonella | 50S rRNA | F | GTAGTACGATGGCGAAACTGC | 5 |
| | | R | CTTCTCGACCCGAGGGACTT | 6 |
| | invA | F | CGCGCTTGATGAGCTTTACC | 7 |
| | | R | CTCGTAATTCGCCGCCATTG | 8 |
| | invE | F | CTTATTGCGCGCCAGCTATC | 9 |
| | | R | CAGCGGAGCGCAACATTTTA | 10 |
| | invF | F | TCGCCAAACGTCACGTAGAA | 11 |
| | | R | CATCCCGTGTATAACCCCCG | 12 |
| | pipB | F | CCAGGGAGGCATGAATCTTA | 13 |
| | | R | TCTTCCAGACAGGCGTTCTT | 14 |
| | sifA | F | TGGCGATGTGTGGATTAAAA | 15 |
| | | R | AAAATGGCGTGAAAAACCTG | 16 |
| | sseA | F | AATCCGGGCTAAGGTGAGTC | 17 |
| | | R | GGGGCTTGAGCATTAAGTTG | 18 |
| | sseJ | F | GGGATTCCCGATTTGTCTTT | 19 |
| | | R | CCGCCTCCATTATCACCTTA | 20 |
| | sspH2 | F | GCACAACTGGCTGAAGATGA | 21 |
| | | R | TTTCCCAGACGGAACATCTC | 22 |
| | 13 | F | ATAATGGCGGTGCGTTTAAG | 23 |
| | | R | TCGAGTCTGTGCCGATGTAG | 24 |
| | 19 | F | ATAGCAGGGGGATACCAACC | 25 |
| | | R | AACTGGCGAAGAACGGTAGA | 26 |
| HD-11 | GAPDH | F | GGTGGTGCTAAGCGTGTTAT | 27 |
| | | R | ACCTCTGTCATCTCTCCACA | 28 |
| | IL-1ß | F | GCTCTACATGTCGTGTGTGATGAG | 29 |
| | | R | TGTCGATGTCCCGCATGA | 30 |
| | IL-6 | F | GCTCGCCGGCTTCGA | 31 |
| | | R | GGTAGGCTGAAAGGCGAACAG | 32 |
| | IL-8 | F | TGGCACCGCAGCTCATT | 33 |
| | | R | TCTTTACCAGCGTCCTACCTTGCGACA | 34 |
| | IL-10 | F | CATGCTGCTGGGCCTGAA | 35 |
| | | R | CGTCTCCTTGATCTGCTTGATG | 36 |
| | IL-18 | F | GGAATGCGATGCCTTTTG | 37 |
| | | R | ATTTTCCCATGCTCTTTCTCA | 38 |
| | iNOS | F | TTGGAAACCAAAGTGTGTAATATCTTG | 39 |
| | | R | CCCTGGCCATGCGTACAT | 40 |
| | LITAF | F | GAGCGTTGACTTGGCTGTC | 41 |
| | | R | AAGCAACAACCAGCTATGCAC | 42 |
| | TLR3 | F | GCAACACTTCATTGAATAGCCTTGAT | 43 |
| | | R | GCCAAACAGATTTCCAATTGCATGT | 44 |
| | TLR7 | F | GGCTGTGAATGAATGGGTGA | 45 |
| | | R | GCTGAATGCTCTGGGAAAGG | 46 |

Based on the description above it will be recognized that LT2 was used as the template strain for developing the described Salmonella live vaccine strain. Salmonella-containing vacuole was formed after invasion, in which Salmonella multiply and survive by expressing SPI-2 genes (Fabrega & Vila, 2013; LaRock, Chaudhary, & Miller, 2015). sseA is one of the SPI-2 genes (Braukmann, Methner, & Berndt, 2015) essential for translocation of effector chaperone proteins of type III secretion system (Coombes, Brown, Kujat-Choy, Vallance, & Finlay, 2003). Genes 13 and 19 of bacteriophage P22, encoding holin and lysozyme, are effectors for Salmonella lysis; holin protein disrupts the bacterial cell membrane by forming pores and lysozyme degrades the peptidoglycan layer of the bacterial cell wall (Vander Byl & Kropinski, 2000). Accordingly, we analyzed whether genes 13 and 19 expression controlled by sseA promoter in Salmonella vaccine strain can lead to intracellular lysis.

The in vitro growth of both STLT2^{+P13+19} and LT2 strains entered log phase after 2-hr incubation and reached stationary phase after 8-hr incubation, indicating that genes 13 and 19 have no effect on the growth of STLT2^{+P13+19}. On the contrary, the intracellular viability of STLT2^{+P13+19} was 94% less than the wild type due to the expression of genes 13 and 19, which indicate that the expression of genes 13 and 19 was induced by sseA promoter in intracellular condition.

TLR3 and TLR7 are generally localized to the endosome membrane surface once stimulated by microbial and host-derived nucleic acids (Lee & Barton, 2014), so as the higher mRNA expressions of TLR3 and TLR7 in STLT2^{+P13+19}-infected HD11 cells serve as evidence for the intracellular autolysis of STLT2^{+P13+19} (O'Neill, Golenbock, & Bowie, 2013). Previously, it was shown that TLR7 can recognize S. typhimurium in infected bone marrow-derived macrophages (Arpaia et al., 2011), while TLR3 can be induced by lipopolysaccharides from Salmonella (Heinz et al., 2003), indicating that the lysis of STLT2^{+P13+19} in Salmonella-containing vacuole could induce and stimulate endosomal TLRs and further activate downstream signaling pathways. Meanwhile, STLT2^{+P13+19} also induced higher mRNA levels of crucial inflammatory cytokines in HD11 cells, suggesting that STLT2^{+P13+19} lysate could activate or differentiate T and B cells, which may further lead to effector cellular immune responses and antibody production against Salmonella in hosts.

Previously, live-attenuated vaccines derived from S. typhimurium have achieved various levels of effectiveness in reducing Salmonella shedding and colonization in poultry. For example, χ9241-tHP oral vaccination at days 1 and 7 could reduce ˜2 logs S. typhimurium in chicken feces and cecum (Jiang et al., 2010); the vaccinated hens with Megan VAC-1 strain were associated with ˜25% lower prevalence of cecal Salmonella (Dorea et al., 2010); Vaxsafe® ST administration reduced ˜50% of S. typhimurium fecal shedding in laying hens (Sharma et al., 2018). In comparison with the abovementioned vaccines, STLT2^{+P13+19} as described herein is more efficient and effective by eliminating 100% STY fecal shedding and intestinal colonization, as well as markedly reducing entire Salmonella prevalence. The production of NO that possesses bactericidal activity serves as a crucial mediator for inflammatory responses (Singh et al., 2012), therefore, we selected iNOS as the general indicator of chicken innate immunity activation against intracellular pathogens. In addition to iNOS, we also detected the upregulated levels of other important pro-inflammatory cytokines IL-6, IL-8, IL-12, and TNF-α (Xu et al., 2019), demonstrating the efficacy of STLT2^{+P13+19} vaccination in stimulating inflammatory responses against Salmonella. On the other hand, based on metagenomic analysis, the modulated gut intestinal microbial composition could be potentially linked with the findings from ELISA. Unique influences of recombinant attenuated Salmonella vaccine on chicken gut microbiota have been previously reported (Redweik, Daniels, Severin, Lyte, & Mellata, 2019). In this disclosure, Salmonella challenge induced gut microbial dysbiosis, exhibited by the lower abundance of Firmicutes and higher abundance of Proteobacteria (Khan & Chousalkar, 2020). STLT2^{+P13+19} effectively shaped gut microbiota by restoring Firmicutes (e.g., Lactobacillus and Bifidobacterium) and reducing Proteobacteria, especially Salmonella, associating with the stimulated inflammatory cytokines.

Thus, the disclosure reveals that the described intracellular autolysis Salmonella strain, STLT2^{+P13+19} can be used as a Salmonella vaccine strain. It carries the lysis genes 13 and 19 of bacteriophage P22 transcriptionally controlled by sseA promoter. The bacterial growth, viability, and adherence onto HD11 cells of STLT2^{+P13+19} are similar to those of the wild type. In contrast, STLT2^{+P13+19} has a greatly reduced intracellular viability as well as significantly induced inflammatory cytokines and endosomal TLRs in HD11 cells. Based on in vivo model, STLT2^{+P13+19} vaccination in chicken significantly reduces Salmonella (especially STY) intestinal colonization, stimulates pro-inflammatory responses, and modulates gut microbial composition. Therefore, the disclosure demonstrates use of STLT2^{+P13+19} as a Salmonella vaccine strain in chicken, which may contribute to reducing the risk of chicken meat contamination, thus preventing human foodborne salmonellosis.

The documents listed below and referenced herein are incorporated herein by reference in their entireties, except for any statements contradictory to the express disclosure herein, subject matter disclaimers or disavowals, and except to the extent that the incorporated material is inconsistent with the express disclosure herein, in which case the language in this disclosure controls. Incorporation by reference of the following shall not be considered an admission by the applicant that the incorporated materials are prior art to the present disclosure, nor shall any document be considered material to patentability of the present disclosure.

### REFERENCES

Agasan, A., Kornblum, J., Williams, G., Pratt, C. C., Fleckenstein, P., Wong, M., & Ramon, A. (2002). Profile of Salmonella enterica subsp. enterica (subspecies I) serotype 4,5,12:i:-strains causing food-borne infections in New York City. J Clin Microbiol, 40(6), 1924-1929. doi:10.1128/jcm.40.6.1924-1929.2002
Anca M. Galis, C. M., Didier Marlier, Daniel Portetelle, Ilie Van, Yves Beckers, and Andr'e Th'ewis. (2013). Control of Salmonella Contamination of Shell Eggs—Preharvest and Postharvest Methods: A Review. Comprehensive Reviews in Food Science and Food Safety, 12, 155-182. doi:10.1111/1541-4337.12007
Antunes, P., Mourao, J., Campos, J., & Peixe, L. (2016). Salmonellosis: the role of poultry meat. Clin Microbiol Infect, 22(2), 110-121. doi:10.1016/j.cmi.2015.12.004
Arpaia, N., Godec, J., Lau, L., Sivick, K. E., McLaughlin, L. M., Jones, M. B., . . . Barton, G. M. (2011). TLR signaling is required for Salmonella typhimurium virulence. Cell, 144(5), 675-688. doi:10.1016/j.cell.2011.01.031
Arsenault, R. J., Napper, S., & Kogut, M. H. (2013). Salmonella enterica Typhimurium infection causes metabolic changes in chicken muscle involving AMPK, fatty acid and insulin/mTOR signaling. Vet Res, 44, 35. doi:10.1186/1297-9716-44-35
Basler, C., Nguyen, T. A., Anderson, T. C., Hancock, T., & Behravesh, C. B. (2016). Outbreaks of Human Salmonella Infections Associated with Live Poultry, United States, 1990-2014. Emerg Infect Dis, 22(10), 1705-1711. doi:10.3201/eid2210.150765
Bradley L. BEARSON, S. M. B. (2015). A. R. B. T. S. O. A. The United States Of America.
Braukmann, M., Methner, U., & Berndt, A. (2015). Immune reaction and survivability of Salmonella typhimurium and salmonella infantis after infection of primary avian macrophages. PLoS One, 10(3), e0122540. doi:10.1371/journal.pone.0122540
CDC. (2013). ANTIBIOTIC RESISTANCE THREATS in the United States. 2013. Retrieved from www.cdc.gov/drugresistance/pdf/ar-threats-2013-508.pdf
CDC. (2014). Surveillance for Foodborne Disease Outbreaks United States, 2014: Annual Report. Retrieved from www.cdc.gov/foodsafety/pdfs/foodborne-outbreaks-annual-report-2014-508.pdf
CDC. (2015). QUICK TIPS FOR PREVENTING SALMONELLA. Retrieved from www.cdc.gov/salmonella/general/prevention.html
Chai, S. J., White, P. L., Lathrop, S. L., Solghan, S. M., Medus, C., McGlinchey, B. M., . . . Mahon, B. E. (2012). Salmonella enterica serotype Enteritidis: increasing incidence of domestically acquired infections. Clin Infect Dis, 54 Suppl 5, S488-497. doi:10.1093/cid/cis231
Coombes, B. K., Brown, N. F., Kujat-Choy, S., Vallance, B. A., & Finlay, B. B. (2003). SseA is required for translocation of Salmonella pathogenicity island-2 effectors into host cells. Microbes Infect, 5(7), 561-570.
da Silva, A. J., Zangirolami, T. C., Novo-Mansur, M. T., Giordano Rde, C., & Martins, E. A. (2014). Live bacterial vaccine vectors: an overview. Braz J Microbiol, 45(4), 1117-1129.
Deguchi, K., Yokoyama, E., Honda, T., & Mizuno, K. (2009). Efficacy of a novel trivalent inactivated vaccine against the shedding of Salmonella in a chicken challenge model. Avian Dis, 53(2), 281-286. doi:10.1637/8516-110908-Reg.1
Detmer, A., & Glenting, J. (2006). Live bacterial vaccines—a review and identification of potential hazards. Microb Cell Fact, 5, 23. doi:10.1186/1475-2859-5-23
Dorea, F. C., Cole, D. J., Hofacre, C., Zamperini, K., Mathis, D., Doyle, M. P., . . . Maurer, J. J. (2010). Effect of Salmonella vaccination of breeder chickens on contamination of broiler chicken carcasses in integrated poultry operations. Appl Environ Microbiol, 76(23), 7820-7825. doi:10.1128/AEM.01320-10
Elanco. (2015). AviPro Megan Vac 1. Retrieved from www.elanco.us/pdfs/2305-24463-megan-vac-broiler-detailer-(for-zm-cert-uspbumv100006)_v05.pdf
Eriksson, S., Lucchini, S., Thompson, A., Rhen, M., & Hinton, J. C. (2003). Unravelling the biology of macrophage infection by gene expression profiling of intracellular Salmonella enterica. Mol Microbiol, 47(1), 103-118.
Fabrega, A., & Vila, J. (2013). Salmonella enterica serovar Typhimurium skills to succeed in the host: virulence and regulation. Clin Microbiol Rev, 26(2), 308-341. doi:10.1128/CMR.00066-12
Goodwin, H., & Shiptsova, R. (2002). Changes in market equilibria resulting from food safety regulation in the meat and poultry industries. The International Food and Agribusiness Management Review 5, 61-74. doi:10.1016/s1096-7508(02)00116-7
Harvey, P. C., Watson, M., Hulme, S., Jones, M. A., Lovell, M., Berchieri, A., Jr., . . . Barrow, P. (2011). Salmonella enterica serovar typhimurium colonizing the lumen of the chicken intestine grows slowly and upregulates a unique set of virulence and metabolism genes. Infect Immun, 79(10), 4105-4121. doi:10.1128/IAI.01390-10
Heinz, S., Haehnel, V., Karaghiosoff, M., Schwarzfischer, L., Muller, M., Krause, S. W., & Rehli, M. (2003). Species-specific regulation of Toll-like receptor 3 genes in men and mice. J Biol Chem, 278(24), 21502-21509. doi:10.1074/jbc.M301476200
Jiang, Y., Kulkarni, R. R., Parreira, V. R., Poppe, C., Roland, K. L., & Prescott, J. F. (2010). Assessment of 2 Salmonella enterica serovar Typhimurium-based vaccines against necrotic enteritis in reducing colonization of chickens by Salmonella serovars of different serogroups. Can J Vet Res, 74(4), 264-270.
Khan, S., & Chousalkar, K. K. (2020). Salmonella typhimurium infection disrupts but continuous feeding of Bacillus based probiotic restores gut microbiota in infected hens. J Anim Sci Biotechnol, 11, 29. doi:10.1186/s40104-020-0433-7
LaRock, D. L., Chaudhary, A., & Miller, S. I. (2015). Salmonellae interactions with host processes. Nat Rev Microbiol, 13(4), 191-205. doi:10.1038/nrmicro3420
Lee, B. L., & Barton, G. M. (2014). Trafficking of endosomal Toll-like receptors. Trends Cell Biol, 24(6), 360-369. doi:10.1016/j.tcb.2013.12.002
Levantesi, C., Bonadonna, L., Briancesco, R., Grohmann, E., Toze, S., & Tandoi, V. (2012). Salmonella in surface and drinking water: Occurrence and water-mediated transmission. Food Research International, 45(2), 587-602. doi:10.1016/j.foodres.2011.06.037
McKee, S. (2012). Salmonella control in poultry processing. Paper presented at the 65th Annual Reciprocal Meat Conference.
Michel, A., Clermont, O., Denamur, E., & Tenaillon, O. (2010). Bacteriophage PhiX174's ecological niche and the flexibility of its Escherichia coli lipopolysaccharide receptor. Appl Environ Microbiol, 76(21), 7310-7313. doi:10.1128/AEM.02721-09
O'Neill, L. A., Golenbock, D., & Bowie, A. G. (2013). The history of Toll-like receptors—redefining innate immunity. Nat Rev Immunol, 13(6), 453-460. doi:10.1038/nri3446
Okamura, M., Tachizaki, H., Kubo, T., Kikuchi, S., Suzuki, A., Takehara, K., & Nakamura, M. (2007). Comparative evaluation of a bivalent killed Salmonella vaccine to prevent egg contamination with Salmonella enterica serovars Enteritidis, Typhimurium, and Gallinarum biovar Pullorum, using 4 different challenge models. Vaccine, 25(25), 4837-4844. doi:10.1016/j.vaccine.2007.03.004
Philippe, N., Alcaraz, J. P., Coursange, E., Geiselmann, J., & Schneider, D. (2004). Improvement of pCVD442, a suicide plasmid for gene allele exchange in bacteria. Plasmid, 51(3), 246-255. doi:10.1016/j.plasmid.2004.02.003
Redweik, G. A. J., Daniels, K., Severin, A. J., Lyte, M., & Mellata, M. (2019). Oral Treatments With Probiotics and Live Salmonella Vaccine Induce Unique Changes in Gut Neurochemicals and Microbiome in Chickens. Front Microbiol, 10, 3064. doi:10.3389/fmicb.2019.03064
Ricke, S. C., Dunkley, C. S., & Durant, J. A. (2013). A review on development of novel strategies for controlling Salmonella Enteritidis colonization in laying hens: fiber-based molt diets. Poult Sci, 92(2), 502-525. doi:10.3382/ps.2012-02763
Scallan, E., Griffin, P. M., Angulo, F. J., Tauxe, R. V., & Hoekstra, R. M. (2011). Foodborne illness acquired in the United States—unspecified agents. Emerg Infect Dis, 17(1), 16-22. doi:10.3201/eid1701.091101p2
Scallan, E., Hoekstra, R. M., Angulo, F. J., Tauxe, R. V., Widdowson, M. A., Roy, S. L., . . . Griffin, P. M. (2011). Foodborne illness acquired in the United States—major pathogens. Emerg Infect Dis, 17(1), 7-15. doi:10.3201/eid1701.P1110110.3201/eid1701.091101p1
Sharma, P., Caraguel, C., Sexton, M., McWhorter, A., Underwood, G., Holden, K., & Chousalkar, K. (2018). Shedding of Salmonella typhimurium in vaccinated and unvaccinated hens during early lay in field conditions: a randomised controlled trial. BMC Microbiol, 18(1), 78. doi:10.1186/s12866-018-1201-0
Shivaprasad, H. L. (2000). Fowl typhoid and pullorum disease. Rev Sci Tech, 19(2), 405-424.
Singh, R., Jain, P., Pandey, N. K., Saxena, V. K., Saxena, M., Singh, K. B., . . . Singh, R. P. (2012). Cytokines Expression and Nitric Oxide Production under Induced Infection to Salmonella typhimurium in Chicken Lines Divergently Selected for Cutaneous Hypersensitivity. Asian-Australas J Anim Sci, 25(7), 1038-1044. doi:10.5713/ajas.2011.11324
Van Gerven, N., Derous, V., & Hernalsteens, J. P. (2008). Expression of in vivo-inducible Salmonella enterica promoters during infection of Caenorhabditis elegans. FEMS Microbiol Lett, 278(2), 236-241. doi:10.1111/j.1574-6968.2007.01001.x
Vander Byl, C., & Kropinski, A. M. (2000). Sequence of the genome of Salmonella bacteriophage P22. J Bacteriol, 182(22), 6472-6481.
Volkova, V. V., Bailey, R. H., & Wills, R. W. (2009). Salmonella in broiler litter and properties of soil at farm location. PLoS One, 4(7), e6403. doi:10.1371/journal.pone.0006403
Whiley, H., & Ross, K. (2015). Salmonella and eggs: from production to plate. Int J Environ Res Public Health, 12(3), 2543-2556. doi:10.3390/ijerph120302543
Wigley, P. (2014). Salmonella enterica in the Chicken: How it has Helped Our Understanding of Immunology in a Non-Biomedical Model Species. Front Immunol, 5, 482. doi:10.3389/fimmu.2014.00482
Wu, H. J., & Wu, E. (2012). The role of gut microbiota in immune homeostasis and autoimmunity. Gut Microbes, 3(1), 4-14. doi:10.4161/gmic.19320
Xu, Z. Y., Yu, Y., Liu, Y., Ou, C. B., Zhang, Y. H., Liu, T. Y., . . . Ma, J. Y. (2019). Differential expression of pro-inflammatory and anti-inflammatory genes of layer chicken bursa after experimental infection with infectious bursal disease virus. Poult Sci, 98(11), 5307-5314. doi:10.3382/ps/pez312

The foregoing Examples are provided for illustration purposes, but do not limit the scope of the disclosure.

1. Modified bacteria wherein the genomes of the bacteria are modified to encode a holin protein and to encode a lysozyme.

2. The modified bacteria of claim 1, wherein the bacteria are non-pathogenic.

3. The modified bacteria of claim 2, wherein the bacteria are non-pathogenic Salmonella.

4. The modified bacteria of claim 3, wherein the non-pathogenic bacteria are Salmonella enterica (SE).

5. The modified bacteria of claim 4, wherein the SE are SE intracellular autolytic SE serovar Typhimurium (S. typhimurium).

6. The modified bacteria claim 5 wherein the holin protein is encoded by gene 13 of Salmonella typhimurium-specific bacteriophage P22 and the lysozyme is encoded by gene 19 of the S. typhimurium-specific bacteriophage P22.

7. The modified bacteria of claim 6, wherein genes encoding the holin protein and the lysozyme are operatively linked to an endogenous bacterial promoter.

8. The modified bacteria of claim 7, wherein the endogenous bacterial promoter is an sseA promoter.

9. The modified bacteria of claim 8, wherein the genes encoding the holin protein and the lysozyme are introduced into chromosomes of the bacteria downstream of the endogenous sseA promoter.

10. The modified bacteria of claim 9, wherein sseA promoted expression of the holin protein and the lysozyme results in intracellular autolysis.

11. A method comprising introducing into an animal in need thereof an effective amount of modified bacteria of claim 1, and wherein said introducing has a prophylactic or therapeutic effect against a bacterial infection in the animal.

12. The method of claim 11, wherein the modified bacteria are introduced into the individual orally.

13. The method of claim 12, wherein introducing the modified bacteria stimulates production of inflammatory cytokines in the individual.

14. The method of claim 13, wherein introducing the modified bacteria reduces Salmonella in the individual.

15. The method of claim 14, wherein reducing the Salmonella comprises reducing Salmonella gut colonization, fecal shedding of Salmonella, or a combination thereof.

16. The method of claim 15, wherein the individual is an avian animal.

17. A vaccine formulation comprising the modified bacteria of claim 1.

18. An article of manufacture comprising the modified bacteria of claim 1, the article of manufacture comprising a sealable container in which the modified bacteria are held.

19. The article of manufacture of claim 18, wherein the modified bacteria are cryopreserved or lyophilized.

20. The article of manufacture of claim 18, the article of manufacture further comprising printed material that provides instructions for administering the modified bacteria to an individual in need thereof.


SEQUENCE LISTING (47 sequences)

SEQ ID NO: 1 - protein, 108 aa, Salmonella Typhimurium-specific bacteriophage P22
MKKMPEKHDLLTAMMAAKEQGIGAILAFAMAYLRGRYNGGAFKKTLIDATMCAIIAWFIRDLLVFAGLSSNLAYIASVFIGYIGTDSIGSLIKRFAAKKAGVDDANQQ

SEQ ID NO: 2 - protein, 146 aa, Typhimurium-specific bacteriophage P22
MMQISSNGITRLKREEGERLKAYSDSRGIPTIGVGHTGKVDGNSVASGMTITAEKSSELLKEDLQWVEDAISSLVRVPLNQNQYDALCSLIFNIGKSAFAGSTVLRQLNLKNYQAAADAFLLWKKAGKDPDILLPRRRRERALFLS

SEQ ID NO: 3 - DNA, 334 bp, Salmonella Typhimurium
gaggggaatgatgataaagaaaaaggctgcgtttagtgaatatcgtgatttagagcaaagttacatgcagctaaatcactgtcttaaaaaatttcaccaaatccgggctaaggtgagtcaacagcttgctgaaagggcagagagccccaaaaatagcagagagacagagagtattcttcataacctatttccacaaggcgttgccggggttaaccaggaggccgagaaggatttaaagaaaatagtaagtttgtttaaacaacttgaagtacgactgaaacaacttaatgctcaagccccggtggagataccgtcaggaaaaacaaaaaggtaa

SEQ ID NO: 4 - protein, 108 aa, Salmonella Typhimurium
MMIKKKAAFSEYRDLEQSYMQLNHCLKKFHQIRAKVSQQLAERAESPKNSRETESILHNLFPQGVAGVNQEAEKDLKKIVSLFKQLEVRLKQLNAQAPVEIPSGKTKR

SEQ ID NO: 5 - DNA, 21 bp, Artificial Sequence
  Primer
gtagtacgatggcgaaactgc

SEQ ID NO: 6 - DNA, 20 bp, Artificial Sequence
  Primer
cttctcgacccgagggactt

SEQ ID NO: 7 - DNA, 20 bp, Artificial Sequence
  Primer
cgcgcttgatgagctttacc

SEQ ID NO: 8 - DNA, 20 bp, Artificial Sequence
  Primer
ctcgtaattcgccgccattg

SEQ ID NO: 9 - DNA, 20 bp, Artificial Sequence
  Primer
cttattgcgcgccagctatc

SEQ ID NO: 10 - DNA, 20 bp, Artificial Sequence
  Primer
cagcggagcgcaacatttta

SEQ ID NO: 11 - DNA, 20 bp, Artificial Sequence
  Primer
tcgccaaacgtcacgtagaa

SEQ ID NO: 12 - DNA, 20 bp, Artificial Sequence
  Primer
catcccgtgtataacccccg

SEQ ID NO: 13 - DNA, 20 bp, Artificial Sequence
  Primer
ccagggaggcatgaatctta

SEQ ID NO: 14 - DNA, 20 bp, Artificial Sequence
  Primer
tcttccagacaggcgttctt

SEQ ID NO: 15 - DNA, 20 bp, Artificial Sequence
  Primer
tggcgatgtgtggattaaaa

SEQ ID NO: 16 - DNA, 20 bp, Artificial Sequence
  Primer
aaaatggcgtgaaaaacctg

SEQ ID NO: 17 - DNA, 20 bp, Artificial Sequence
  Primer
aatccgggctaaggtgagtc

SEQ ID NO: 18 - DNA, 20 bp, Artificial Sequence
  Primer
ggggcttgagcattaagttg

SEQ ID NO: 19 - DNA, 20 bp, Artificial Sequence
  Primer
gggattcccgatttgtcttt

SEQ ID NO: 20 - DNA, 20 bp, Artificial Sequence
  Primer
ccgcctccattatcacctta

SEQ ID NO: 21 - DNA, 20 bp, Artificial Sequence
  Primer
gcacaactggctgaagatga

SEQ ID NO: 22 - DNA, 20 bp, Artificial Sequence
  Primer
tttcccagacggaacatctc

SEQ ID NO: 23 - DNA, 20 bp, Artificial Sequence
  Primer
ataatggcggtgcgtttaag

SEQ ID NO: 24 - DNA, 20 bp, Artificial Sequence
  Primer
tcgagtctgtgccgatgtag

SEQ ID NO: 25 - DNA, 20 bp, Artificial Sequence
  Primer
atagcagggggataccaacc

SEQ ID NO: 26 - DNA, 20 bp, artificial sequence
  Primer
aactggcgaagaacggtaga

SEQ ID NO: 27 - DNA, 20 bp, Artificial Sequence
  Primer
ggtggtgctaagcgtgttat

SEQ ID NO: 28 - DNA, 20 bp, Artificial Sequence
  Primer
acctctgtcatctctccaca

SEQ ID NO: 29 - DNA, 24 bp, Artificial Sequence
  Primer
gctctacatgtcgtgtgtgatgag

SEQ ID NO: 30 - DNA, 18 bp, Artificial Sequence
  Primer
tgtcgatgtcccgcatga

SEQ ID NO: 31 - DNA, 15 bp, Artificial Sequence
  Primer
gctcgccggcttcga

SEQ ID NO: 32 - DNA, 21 bp, Artificial Sequence
  Primer
ggtaggctgaaaggcgaacag

SEQ ID NO: 33 - DNA, 17 bp, Artificial Sequence
  Primer
tggcaccgcagctcatt

SEQ ID NO: 34 - DNA, 27 bp, Artificial Sequence
  Primer
tctttaccagcgtcctaccttgcgaca

SEQ ID NO: 35 - DNA, 18 bp, Artificial Sequence
  Primer
catgctgctgggcctgaa

SEQ ID NO: 36 - DNA, 22 bp, Artificial Sequence
  Primer
cgtctccttgatctgcttgatg

SEQ ID NO: 37 - DNA, 18 bp, Artificial Sequence
  Primer
ggaatgcgatgccttttg

SEQ ID NO: 38 - DNA, 21 bp, Artificial Sequence
  Primer
attttcccatgctctttctca

SEQ ID NO: 39 - DNA, 27 bp, Artificial Sequence
  Primer
ttggaaaccaaagtgtgtaatatcttg

SEQ ID NO: 40 - DNA, 18 bp, Artificial Sequence
  Primer
ccctggccatgcgtacat

SEQ ID NO: 41 - DNA, 19 bp, Artificial Sequence
  Primer
gagcgttgacttggctgtc

SEQ ID NO: 42 - DNA, 21 bp, Artificial Sequence
  Primer
aagcaacaaccagctatgcac

SEQ ID NO: 43 - DNA, 26 bp, Artificial Sequence
  Primer
gcaacacttcattgaatagccttgat

SEQ ID NO: 44 - DNA, 25 bp, Artificial Sequence
  Primer
gccaaacagatttccaattgcatgt

SEQ ID NO: 45 - DNA, 20 bp, Artificial Sequence
  Primer
ggctgtgaatgaatgggtga

SEQ ID NO: 46 - DNA, 20 bp, Artificial Sequence
  Primer
gctgaatgctctgggaaagg

SEQ ID NO: 47 - DNA, 34 bp, Salmonella Typhimurium
gactcgctacgctcgcccttcgggccgccgctag